(19) United States
(12) Patent Application Publication
(10) Pub. No.: US 2026/0327650 A1
(43) Pub. Date: Oct. 22, 2026

(54) DUAL TRANSFECTION VECTOR

(71) Applicant: Solid Biosciences Inc., Charlestown, MA (US)

(72) Inventor: Sarath Chandra Mandava, Malden, MA (US)

(21) Appl. No.: 19/117,448

(22) PCT Filed: Oct. 5, 2023
(86) PCT No.: PCT/US2023/034596

§ 371 Date: Apr. 1, 2025

Related U.S. Application Data
(60) Provisional application No. 63/413,887, filed on Oct. 6, 2022.

(51) Int. Cl. C12N 15/86

(52) U.S. Cl. CPC C12N 15/86; C12N 2750/14143; C12N 2750/14151

(57) ABSTRACT

The invention described herein provides an improved system and method for rAAV production. The system of the invention includes a recombinant DNA vector that comprises both the gene of interest (GOI) to be delivered to a target cell, as well as the Rep and Cap coding sequences for rAAV packaging.

### REFERENCE TO RELATED APPLICATION

This application claims priority to and the benefit of the filing date of U.S. Provisional Application No. 63/413,887, filed on Oct. 6, 2022, the entire contents of which are incorporated herein by reference.

### BACKGROUND OF THE INVENTION

In the early days of rAAV production, cultured cells are first infected with adenovirus, then transfected with two additional DNA plasmids: one plasmid containing a pair of essential rAAV genes (e.g., Rep/Cap genes), and the other plasmid carrying a sequence of a gene of interest (GOI). This method produces a reasonable yield of rAAV particles, but there is a risk of adenovirus contaminating the preparation which can put patients treated with rAAV at risk.

In 1998, different research groups determined that the key genes of the adenovirus can be shifted into a third “helper” plasmid, and all three plasmids can be transfected into a preferred host cell at the same time to produce rAAV particles, circumventing the potential contamination of rAAV. Since then, this method known as the “triple transfection” (TT) technique has been widely used as the main method to produce recombinant adeno associated virus (rAAV), including commercial manufacturing of AAV viral stocks for gene therapy, and also may be referred herein as traditional triple transfection or conventional triple transfection.

However, a persistent problem of using AAV-based gene therapy is the demand for large quantities of suitable AAV viral particles (e.g., in the range of about 1×10¹⁴ to 1× 1015 vector genome) for efficacious treatment.

Today, rAAV is considered a key platform to deliver numerous types of genes for gene therapy, and the number of rAAV-based therapies in development continues to increase. Therefore, there is a need for further improving large scale rAAV production.

### SUMMARY OF THE INVENTION

One aspect of the invention provides a recombinant DNA vector comprising: (a) a gene-of-interest (GOI) flanked by a 5′ adeno-associated virus (AAV) ITR (such as AAV2 5′ ITR) sequence and a 3′ ITR sequence (such as AAV2 3′ ITR); (b) a coding sequence for an AAV Rep (such as AAV2 Rep, or Rep2) compatible with said 5′ AAV ITR and said 3′ AAV ITR, optionally, said coding sequence for the AAV Rep is under the transcriptional control of an AAV P5 promoter positioned downstream or 3′ to the GOI; and, (c) a coding sequence for an AAV Cap; wherein expression of said AAV Rep, in a host cell comprising AAV helper genes, is sufficient to package an AAV vector genome (vg) comprising the GOI flanked by the 5′- and 3-ITR sequences into an AAV capsid comprising said AAV Cap, with the proviso that the recombinant DNA vector is not a Herpes Simplex Virus (HSV) vector.

Another aspect of the invention provides a vector comprising (a) a gene-of-interest (GOI) flanked by adeno-associated virus (AAV) ITRs; (b) a coding sequence for an AAV Rep (such as AAV2 Rep, or Rep2) compatible with said ITRs, optionally, said coding sequence for the AAV Rep is under the transcriptional control of an AAV P5 promoter positioned downstream or 3′ to the GOI; and, (c) a coding sequence for an AAV Cap; wherein the vector is not a Herpes Simplex Virus (HSV) vector.

In certain embodiments, the recombinant DNA vector, or vector, is a plasmid.

In another aspect, the invention provides a plasmid comprising (a) a gene-of-interest (GOI) flanked by (AAV) ITRs (such as an AAV2 5′ ITR sequence and an AAV2 3′ ITR sequence); (b) a coding sequence for an AAV Rep (such as AAV2 Rep, or Rep2) compatible with said (AAV) ITRs, optionally, said coding sequence for the AAV Rep is under the transcriptional control of an AAV P5 promoter positioned downstream or 3′ to the GOI; and (c) a coding sequence for an AAV Cap.

In another aspect, the invention provides a plasmid comprising (a) a gene-of-interest (GOI) flanked by a 5′ adeno-associated virus (AAV) ITR (such as AAV2 5′ ITR) sequence and a 3′ ITR sequence (such as AAV2 3′ ITR); (b) a coding sequence for an AAV Rep (such as AAV2 Rep, or Rep2) compatible with said 5′ AAV ITR and said 3′ AAV ITR, optionally, said coding sequence for the AAV Rep is under the transcriptional control of an AAV P5 promoter positioned downstream or 3′ to the GOI; and, (c) a coding sequence for an AAV Cap; wherein expression of said AAV Rep, in a host cell comprising AAV helper genes, is sufficient to package an AAV vector genome (vg) comprising the GOI flanked by the 5′- and 3-ITR sequences into an AAV capsid comprising said AAV Cap.

In certain embodiments, the plasmid of the invention does not comprise a polynucleotide sequence encoding a helper gene.

In yet another aspect, the invention provides a two-plasmid system comprising a vector plasmid and a helper plasmid, wherein the vector plasmid comprises a GOI, a rep gene encoding functional Rep proteins, and a cap gene encoding functional Cap proteins.

In some embodiments, expression of the AAV Rep in a host cell comprising AAV helper genes is sufficient to package an AAV vector genome (vg) comprising the GOI flanked by the 5′- and 3-ITR sequences into an AAV capsid comprising the AAV Cap.

In certain embodiments, the GOI is within a pro-AAV cassette comprising the GOI operably linked to a promoter.

In certain embodiments, the pro-AAV cassette further comprises: (1) an enhancer that promotes the transcription of the GOI from the promoter; (2) a 5′ UTR; (3) a Kozak sequence; (4) a heterologous intron that promotes transcription and/or translation of the GOI; (5) a 3′ UTR; (6) a WPRE sequence; and/or (7) a polyA signal sequence.

In certain embodiments, the coding sequence for the AAV Rep and the coding sequence for the AAV Cap are within a RepCap cassette comprising an operably-linked RepCap promoter (such as the AAV P5 promoter).

In certain embodiments, the RepCap cassette and the pro-AAV cassette: i) are immediately adjacent to each other (e.g., with substantially no intervening polynucleotide sequence); ii) are not immediately adjacent to each other; iii) have the same transcription direction; iv) have opposite transcription direction.

In certain embodiments, the recombinant DNA vector further comprises a bacterial replication Ori gene, a selection marker (such as an antibiotic resistance gene, e.g., KanR or AmpR) under the transcriptional control of a selection marker promoter (such as a bacterial promoter).

In certain embodiments, the GOI is a functional equivalent of dystrophin (e.g., a dystrophin minigene encoding a functional micro-dystrophin protein).

In certain embodiments, the GOI includes a gene responsible for/defective in LGMD2E (limb-girdle muscular dystrophy type 2E), LGMD2D (limb-girdle muscular dystrophy type 2D), LGMD2C (limb-girdle muscular dystrophy type 2C), LGMD2B (limb-girdle muscular dystrophy type 2B), LGMD2L (limb-girdle muscular dystrophy type 2L), LGMD2I (limb-girdle muscular dystrophy type 21), or a gene or coding sequence for NAGLU (α-N-acetylglucosaminidase, for Sanfilippo syndrome or mucopolysaccharidosis type IIIB (MPS IIIB)), sulfamidase or SGSH (for mucopolysaccharidosis type IIIA or MPS IIIA), Factor IX, Factor VIII, Myotubularin 1 (MTM1), Survival of Motor Neuron (SMN, for spinal muscular atrophy or SMA), GalNAc transferase GALGT2, calpain-3 (CAPN-3), acid alpha-glucosidase (GAA, for Pompe disease), alpha-galactosidase A or GLA (for Fabry disease), glucocerebrosidase, dystrophin or microdystrophin.

In certain embodiments, the GOI is a microdystrophin gene (e.g., one described in U.S. Pat. Nos. 7,906,111; 7,001,761; 7,510,867; 6,869,777; 8,501,920; 7,892,824; WO2016115543; WO 2023/018854, or U.S. Pat. No. 10,166,272, each and every one incorporated herein by reference). In certain embodiments, the GOI is a microdystrophin gene having the nucleotide sequence of SEQ ID NO: 1 of WO 2023/018854 (incorporated herein by reference).

In certain embodiments, the microdystrophin gene comprises a coding sequence for R16 and R17 spectrin-like repeats for the full length dystrophin protein (such as one described in U.S. Pat. No. 7,892,824).

In certain embodiments, the microdystrophin gene comprises a coding sequence for the R1, R16, R17, R23, and R24 spectrin-like repeats of the full-length dystrophin protein (such as the microdystrophin gene described in PCT/US2016/013733).

In certain embodiments, the 5′ and 3′ AAV ITR sequences flanking said GOI are both from AAV1, AAV2, AAV3A, AAV3B, AAV4, AAV5, AAV6, AAV7, AAV8, AAV9, AAV10, AAV11, AAV12, AAV13, AAVrh74, or AAV-DJ.

In certain embodiments, the 5′ and 3′ AAV ITR sequences flanking said GOI are both from AAV2.

In certain embodiments, the tropism of the AAV include skeletal muscle (such as AAV1, AAV6, AAV7, AAV8, or AAV9, preferably AAV9).

In certain embodiments, the AAV ITR, said AAV Rep, and said AAV Cap are from the same or different AAVs.

In certain embodiments, the AAV ITR is AAV2 ITR, said AAV Rep is Rep2 from AAV2, and said AAV Cap is Cap9 from AAV9 or a derivative thereof (such as a spectrum or transcription derivative thereof).

In certain embodiments, the coding sequence for AAV Rep and Cap proteins is under the transcriptional control of a promoter, such as an AAV p5 promoter, an upstream HSV promoter, a modified p5 promoter lacking RBE (Rep-Binding Element), an HPV P97 promoter containing a REP binding site and a transcription start site-localized YY1 binding site, or a ubiquitous promoter (such as CMV promoter, EF1a promoter, CAG promoter, CB promoter etc).

In certain embodiments, the modified P5 promoter is a recombinant P5 promoter. In some embodiments, the modified P5 promoter comprises a REP binding site and a transcription start site-localized Ying-Yang 1 (YY1) binding site. In some embodiments, the modified P5 promoter comprises an exogenous spacer sequence inserted between the REP binding site and YY1 binding site. In some embodiments, the spacer is 5 nucleotides to 100 nucleotides in length (e.g., about 5 nts).

In certain embodiments, the modified P5 promoter is a recombinant P5 promoter comprising a REP binding site and a transcription start site-localized Ying-Yang 1 (YY1) binding site, and wherein said modified P5 promoter comprises an exogenous spacer sequence inserted between the REP binding site and YY1 binding site; optionally, the spacer is 5 nucleotides to 100 nucleotides in length (e.g., about 5 nts).

In certain embodiments, the AAV helper genes comprise adenoviral, herpesviral, or papillomaviral genes useful for AAV packaging (such as E1A, E1B, E2A, E4 and VA RNA), optionally operably linked to a promoter as one transcriptional unit.

Another aspect of the invention provides a composition or a kit, comprising the recombinant DNA vector of the invention, and a helper plasmid.

In certain embodiments, the helper plasmid comprises a helper virus gene that sufficiently supports AAV packaging.

In certain embodiments, the helper virus gene comprises: (i) an adenovirus gene, optionally an Adenovirus 5 or Adenovirus 2 gene; and/or (ii) a VA nucleic acid encoding functional VA RNA I and II, an E2A gene encoding a functional E2A protein, and an E4 gene encoding a functional E4 protein.

Another aspect of the invention provides a host cell comprising the recombinant DNA vector of the invention, or the composition of the invention.

In certain embodiments, the host cell is a HEK293 cell (such as an Expi293F cell), a VP2 cell, a Vero cell, an HUH7 cell, a HepG2 cell, a HeLa cell, an A549 cell, a BHK cell, or an insect cell (such as Sf9).

Another aspect of the invention provides a method of propagating/amplifying/producing a recombinant replication-defective AAV viral particle encapsidating the GOI of the invention, the method comprising: introducing the recombinant DNA vector of the invention into a host cell, prior to, concurrently with, or subsequent to introducing said AAV helper genes of the invention to said host cell, thereby propagating/amplifying/producing the recombinant replication-defective AAV viral particle.

In certain embodiments, the method further comprises harvesting the recombinant replication-defective AAV viral particle of the invention from the host cell.

In certain embodiments, the host cell is a HEK293 cell (such as an Expi293F cell), a VP2 cell, a Vero cell, an HUH7 cell, a HepG2 cell, a HeLa cell, an A549 cell, a BHK cell, or an insect cell (such as Sf9).

In certain embodiments, the recombinant DNA vector is introduced to the host cell by transient transfection.

It should be understood that any one embodiment of the invention, including those only described in the Examples, claims, or one of the subsections, can be combined with any other one or more embodiments, unless improper or expressly disclaimed.

### BRIEF DESCRIPTION OF THE DRAWINGS

FIG. 1A shows a schematic (not to scale) drawing of an enlarged section of an exemplary vector of the invention, showing the Pro-AAV cassette and the RepCap cassette (which may or may not be directly adjacent to each other, and which may or may not be in the same transcription orientation, as shown here). The pZ-KanR (kanamycin resistant gene under the transcriptional control of a bacterial promoter) is an optional feature as well. GOI: gene-of-interest.

FIG. 1B shows plasmid maps of two exemplary plasmids comprising the dual transfection GOI/RepCap vectors of the invention to produce AAV9 (left) and AAV-SLB101 (right) viral particles, wherein the GOI is a variant of microdystrophin as a non-limiting example of the GOI. μDys: microdystrophin. In both constructs, the Pro-AAV cassette is positioned upstream/5′ to the RepCap cassette, the transcription of which is driven by the p5 promoter in the RepCap cassette. As a result, in a circular plasmid vector, the KanR coding sequence is further upstream/5′ to the p5 promoter compared to the equivalent KanR coding sequence in the RepCap plasmid used in triple transfection (in which there is no Pro-AAV cassette).

FIG. 2 shows results of rAAV production using Expi cells by dual transfection of the GOI/RepCap plasmid of the invention and a suitable helper plasmid, at an exemplary GOI: Helper (w/w) ratio of 1:1 and 1.5:1, and DNA amount of 0.5 μg/cell and 0.75 μg/cell, respectively.

FIG. 3A shows the results of surprisingly high titer of rAAV viral particle yields (e.g., 200-300%) of AAV9 and AAV-SLB101 viral particles produced in Expi cells, using the dual transfection (DT) plasmid of the invention (see FIG. 1B) as compared to the traditional triple transfection (TT) plasmids. Viral titers were determined at 24, 48 and 72 hours post transfection by ddPCR, by lysing the infected Expi cells.

FIG. 3B shows superior yields of AAV produced by the various methods using the DT plasmids of the invention (the 3^{rd}-5^{th} bar from the left), compared to the triple transfection methods (the first two bars from the left).

FIG. 3C shows scalable high yield production of AAV in 2L bioreactors using the DT plasmids of the invention.

FIG. 4 shows reduced mis-packaging rates of KanR using the DT plasmids of the invention as compared to the TT plasmids, based on ddPCR and KanR/GOI ratio, using two commercially available transfection reagents. AAVMAX: the Gibco AAV-MAX Transfection Kit (ThermoFisher Scientific). FectoVIR: FECTOVIR®-AAV (Polyplus, NY).

FIG. 5 shows that modified p5 promoter further reduces mis-packaging rates (as measured by % of p5-related mis-packaging/GOI) and increases GOI titer.

### DETAILED DESCRIPTION OF THE INVENTION

### 1. Overview

Conventional AAV production system utilize three plasmid vectors (the so-called triple transfection vector system): one with a gene of interest (GOI) flanked by AAV ITR sequences, for packaging into the AAV viral particles; another with an expression cassette encoding the AAV Rep and Cap proteins useful for AAV packaging; and yet another provides the useful helper genes from other viruses (such as adenovirus, herpesvirus, or papillomavirus) for productive AAV life cycle.

The recombinant dual transfection vectors of the invention described herein improve the conventional system by inserting both the GOI cassette and the rep-cap expression cassette into a single plasmid, to create the dual transfection vector (e.g., plasmid) of the invention that can be used in double transfection with the helper plasmid (e.g., the same helper plasmid used in the traditional triple transfection method).

It was surprisingly found that, at suitable (e.g., identical/equal molar) ratios of the Rep, Cap, and helper genes, the dual transfection system of the invention produced rAAV viral particles at about 200-300% level compared to the traditional triple transfection system that has been used in commercial production of rAAV viral particles for decades.

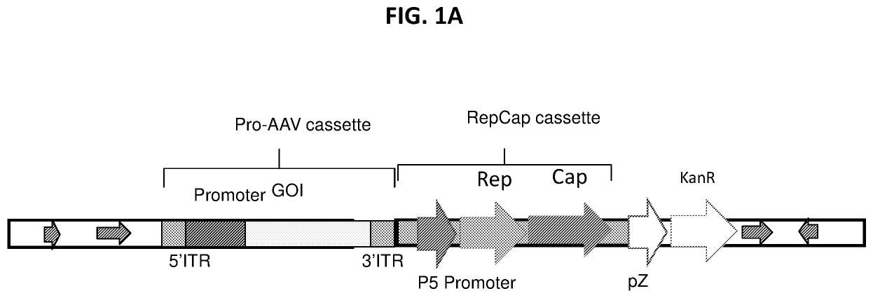

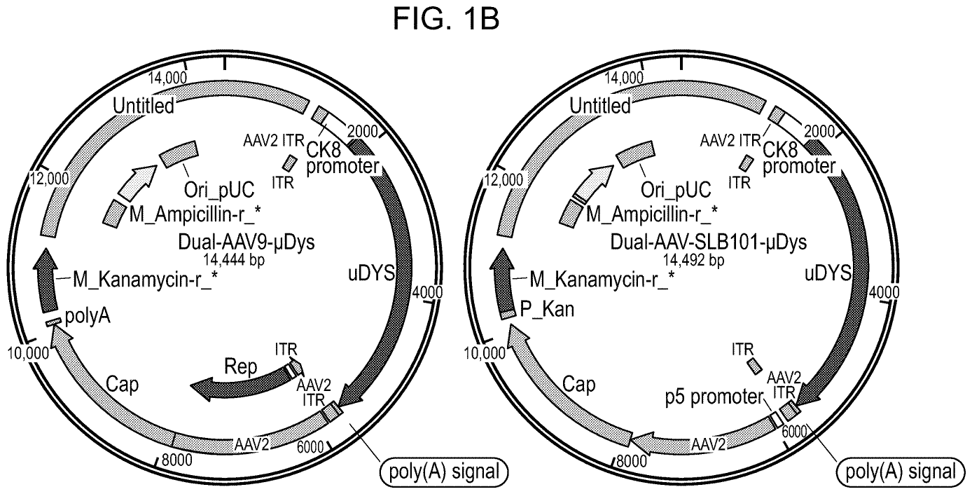

Further, in some embodiments, AAV production using the dual vector/plasmid system of the invention results in reduced frequency of mispackaging. An exemplary such embodiment for the rDNA vector of the invention comprises a configuration as shown in FIG. 1A.

Thus, one aspect of the invention provides a vector comprising (a) a gene-of-interest (GOI) flanked by adeno-associated virus (AAV) ITRs; (b) a coding sequence for an AAV Rep (such as AAV2 Rep, or Rep2) compatible with said ITRs, optionally, said coding sequence for the AAV Rep is under the transcriptional control of an AAV P5 promoter positioned downstream or 3′ to the GOI; and (c) a coding sequence for an AAV Cap; wherein the vector is not a Herpes Simplex Virus (HSV) vector.

Another aspect of the invention provides a recombinant DNA (rDNA) vector comprising: (a) a gene-of-interest (GOI) flanked by a 5′ adeno-associated virus (AAV) ITR (such as AAV2 5′ ITR) sequence and a 3′ ITR sequence (such as AAV2 3′ ITR); (b) a coding sequence for an AAV Rep (such as AAV2 Rep, or Rep2) compatible with said 5′ AAV ITR and said 3′ AAV ITR, optionally, said coding sequence for the AAV Rep is under the transcriptional control of an AAV P5 promoter positioned downstream or 3′ to the GOI; and, (c) a coding sequence for an AAV Cap; wherein expression of the AAV Rep, in a host cell comprising AAV helper genes, is sufficient to package an AAV vector genome (vg) comprising the GOI flanked by the 5′- and 3-ITR sequences into an AAV capsid comprising said AAV Cap, with the proviso that the recombinant DNA vector is not a Herpes Simplex Virus (HSV) vector.

In certain embodiments, the vector or recombinant DNA vector is a plasmid.

In certain embodiments, the vector is suitable for use in a mammalian cell. Vectors suitable for use in mammalian cells are widely described and well-known in the art. Those skilled in the art would appreciate that vectors of the present invention may also contain various additional sequences and elements useful for the replication of the vector in prokaryotic and/or eukaryotic cells, selection of the vector and the expression of the sequences in a variety of host cells. For example, the vectors of the present disclosure may include a prokaryotic replicon (that is, a sequence having the ability to direct autonomous replication and maintenance of the vector extra chromosomally in a prokaryotic host cell, such as a bacterial host, cell. Such replicons are well known in the art. In some embodiments, the vectors may include a shuttle element that makes the vectors suitable for replication and integration in both prokaryotes and eukaryotes. In addition, vectors may also include a gene whose expression confers a detectable marker such as a drug resistance gene, which allows for selection and maintenance of the host cells. Vectors may also have a reportable marker, such as gene encoding a fluorescent or other detectable protein.

The vectors of the invention may also include transcriptional enhancers, translational signals, and transcriptional and translational termination signals. Examples of transcriptional termination signals include, but are not limited to, polyadenylation signal sequences, such as bovine growth hormone (BGH) poly(A), SV40 late poly(A), rabbit beta-globin (RBG) poly(A), thymidine kinase (TK) poly (A) sequences, and any variants thereof.

In certain embodiments, the dual transfection recombinant DNA vector of the invention (the dual transfection (DT) vector) comprises a gene-of-interest (GOI) flanked by a 5′ and 3′ adeno-associated virus (AAV, such as AAV2) ITR, a coding sequence for an AAV Rep (such as AAV2 Rep) and a coding sequence for an AAV Cap (such as AAV9 or derivative thereof) compatible with the AAV ITRs. In a host cell comprising AAV helper genes (which can be introduced into the host cell by any art-recognized means, such as infection of the host cell by adenovirus, or transfection of a helper plasmid comprising the requisite genes for AAV packaging), the expression of the AAV Rep and Cap proteins from the dual transfection vector is sufficient to package an AAV vector genome (vg) comprising the GOI flanked by the 5′ and 3′ AAV ITRs.

In certain embodiments, the recombinant DNA vector is a plasmid, which may be suitable for transfection into an AAV packaging cell line, such as Expi, HEK293 (such as HEK293T cells), or other commonly used AAV packaging cell lines.

In a related aspect, the invention provides a plasmid comprising (a) a gene-of-interest (GOI) flanked by (AAV) ITRs; (b) a coding sequence for an AAV Rep (such as AAV2 Rep, or Rep2) compatible with said (AAV) ITRs, optionally, said coding sequence for the AAV Rep is under the transcriptional control of an AAV P5 promoter positioned downstream or 3′ to the GOI; and (c) a coding sequence for an AAV Cap.

In another related aspect, the invention provides a plasmid comprising (a) a gene-of-interest (GOI) flanked by a 5′ adeno-associated virus (AAV) ITR (such as AAV2 5′ ITR) sequence and a 3′ ITR sequence (such as AAV2 3′ ITR); (b) a coding sequence for an AAV Rep (such as AAV2 Rep, or Rep2) compatible with said 5′ AAV ITR and said 3′ AAV ITR, optionally, said coding sequence for the AAV Rep is under the transcriptional control of an AAV P5 promoter positioned downstream or 3′ to the GOI; and, (c) a coding sequence for an AAV Cap; wherein expression of said AAV Rep, in a host cell comprising AAV helper genes, is sufficient to package an AAV vector genome (vg) comprising the GOI flanked by the 5′- and 3-ITR sequences into an AAV capsid comprising said AAV Cap.

In some embodiments, the plasmid of the invention does not comprise a polynucleotide sequence encoding a helper gene.

In yet another aspect, the invention provides a two-plasmid system comprising a vector plasmid and a helper plasmid, wherein the vector plasmid comprises a GOI, a rep gene encoding functional Rep proteins, and a cap gene encoding functional Cap proteins.

As used herein, the term “plasmid” includes a nucleic acid molecule that can replicate independently of a cell chromosome. The term “plasmid” is intended to include circular nucleic acid molecules and linear nucleic acid molecules. Furthermore, the term “plasmid” is intended to include bacterial plasmids, cosmids, minicircles (Nehlsen et al., Gene Ther. Mol. Biol., 10:233-244, 2006; and Kay et al., Nature Biotechnology, 28:1287-1289, 2010) and ministrings (Nafissi et al., Mol Ther Nucleic Acids, 3: el65, 2014). In certain embodiments, the plasmid is a circular nucleic acid (DNA) molecule. In certain embodiments, the plasmid is a nucleic acid molecule that is of bacterial origin.

In some embodiments, the GOI is within a pro-AAV cassette comprising the GOI operably linked to a promoter.

In certain embodiments, the rDNA vector comprises two or more copies of the pro-AAV cassette. In certain embodiments, all copies of the pro-AAV cassette comprise the same GOI. In certain embodiments, at least two of the pro-AAV cassettes comprise different GOI. The latter embodiment can be useful, for example, if AAV vectors are used to deliver different parts of the same functional assembly, such as a CRISPR/Cas effector enzyme and the coding sequence for one or more guide RNAs.

In certain embodiments, the pro-AAV cassette further comprises: (1) an enhancer that promotes the transcription of the GOI from the promoter; (2) a 5′ UTR; (3) a Kozak sequence; (4) a heterologous intron that promotes transcription and/or translation of the GOI; (5) a 3′ UTR; (6) a WPRE sequence; and/or (7) a polyA signal sequence.

In some embodiments, the coding sequence for the AAV Rep and the coding sequence for the AAV Cap are within a RepCap cassette comprising an operably-linked RepCap promoter. In certain embodiments, the operably-linked RepCap promoter comprises the AAV P5 promoter.

In some embodiments, the RepCap cassette and the pro-AAV cassette are immediately adjacent to each other (e.g., with substantially no intervening polynucleotide sequence).

In some other embodiments, the RepCap cassette and the pro-AAV cassette are not immediately adjacent to each other.

In some embodiments, the RepCap cassette and the pro-AAV cassette have the same transcription direction.

In some other embodiments, the RepCap cassette and the pro-AAV cassette have opposite transcription directions.

In some embodiments, the pro-AAV cassette is upstream of the RepCap cassette.

In some other embodiments, the pro-AAV cassette is downstream of the RepCap cassette.

In some embodiments, the GOI is in front of the coding sequence for the AAV Rep. In some embodiments, the GOI is in front of the coding sequence for the AAV Cap. In some embodiments, the GOI is in front of the coding sequences for both the AAV Rep and AAV Cap.

In certain embodiments, the coding sequence for the AAV Rep is under the transcriptional control of an AAV P5 promoter positioned downstream or 3′ to the GOI.

In some embodiments, the expression of the AAV Rep in a host cell comprising AAV helper genes is sufficient to package an AAV vector genome (vg) comprising the GOI flanked by the 5′- and 3-ITR sequences into an AAV capsid comprising the AAV Cap.

In some embodiments, the recombinant DNA vector (dual transfection vector) further comprises a bacterial replication Ori gene, a selection marker (such as an antibiotic resistance gene, e.g., KanR or AmpR) under the transcriptional control of a selection marker promoter, such as a bacterial promoter.

In certain embodiments, the GOI is a functional equivalent of dystrophin (e.g., a dystrophin minigene encoding a functional micro-dystrophin protein).

In certain embodiments, the GOI includes a gene responsible for/defective in LGMD2E (limb-girdle muscular dystrophy type 2E), LGMD2D (limb-girdle muscular dystrophy type 2D), LGMD2C (limb-girdle muscular dystrophy type 2C), LGMD2B (limb-girdle muscular dystrophy type 2B), LGMD2L (limb-girdle muscular dystrophy type 2L), LGMD2I (limb-girdle muscular dystrophy type 21), or a gene or coding sequence for NAGLU (α-N-acetylglucosaminidase, for Sanfilippo syndrome or mucopolysaccharidosis type IIIB (MPS IIIB)), sulfamidase or SGSH (for mucopolysaccharidosis type IIIA or MPS IIIA), Factor IX, Factor VIII, Myotubularin 1 (MTM1), Survival of Motor Neuron (SMN, for spinal muscular atrophy or SMA), GalNAc transferase GALGT2, calpain-3 (CAPN-3), acid alpha-glucosidase (GAA, for Pompe disease), alpha-galactosidase A or GLA (for Fabry disease), glucocerebrosidase, dystrophin or microdystrophin.

In certain embodiments, the GOI is a microdystrophin gene (e.g., one described in U.S. Pat. Nos. 7,906,111; 7,001,761; 7,510,867; 6,869,777; 8,501,920; 7,892,824; WO2016115543; WO 2023/018854, or U.S. Pat. No. 10,166,272, each and every one incorporated herein by reference). In certain embodiments, the GOI is a microdystrophin gene having the nucleotide sequence of SEQ ID NO: 1 of WO 2023/018854 (incorporated herein by reference).

In certain embodiments, the microdystrophin gene comprises a coding sequence for R16 and R17 spectrin-like repeats for the full length dystrophin protein (such as one described in U.S. Pat. No. 7,892,824).

In certain embodiments, the microdystrophin gene comprises a coding sequence for the R1, R16, R17, R23, and R24 spectrin-like repeats of the full-length dystrophin protein (such as the microdystrophin gene described in PCT/US2016/013733).

In certain embodiments, the 5′ and 3′ AAV ITR sequences flanking said GOI are both from AAV1, AAV2, AAV3A, AAV3B, AAV4, AAV5, AAV6, AAV7, AAV8, AAV9, AAV10, AAV11, AAV12, AAV13, AAVrh74, or AAV-DJ.

In certain embodiments, the tropism of the AAV include skeletal muscle (such as AAV1, AAV6, AAV7, AAV8, or AAV9, preferably AAV9).

In certain embodiments, the AAV ITR, the AAV Rep, and the AAV Cap are from the same or different AAVs.

In certain embodiments, the AAV ITR is AAV2 ITR, the AAV Rep is Rep2 from AAV2, and the AAV Cap is Cap9 from AAV9 or a derivative thereof (such as a spectrum or transcription derivative thereof).

In certain embodiments, the coding sequence for AAV Rep and Cap proteins is under the transcriptional control of a promoter, such as an AAV p5 promoter, a modified p5 promoter lacking RBE (Rep-Binding Element), an HPV P97 promoter containing a REP binding site and a transcription start site-localized YY1 binding site, or a ubiquitous promoter (such as CMV promoter, EF1a promoter, CAG promoter, CB promoter etc).

In certain embodiments, the modified P5 promoter is a recombinant P5 promoter. In some embodiments, the modified P5 promoter comprises a REP binding site and a transcription start site-localized Ying-Yang 1 (YY1) binding site. In some embodiments, the modified P5 promoter comprises an exogenous spacer sequence inserted between the REP binding site and YY1 binding site. In some embodiments, the spacer is 5 nucleotides to 100 nucleotides in length (e.g., about 5 nts).

In certain embodiments, the modified P5 promoter is a recombinant P5 promoter comprising a REP binding site and a transcription start site-localized Ying-Yang 1 (YY1) binding site, and wherein said modified P5 promoter comprises an exogenous spacer sequence inserted between the REP binding site and YY1 binding site; optionally, the spacer is 5 nucleotides to 100 nucleotides in length (e.g., about 5 nts).

In certain embodiments, the AAV helper genes comprise adenoviral, herpesviral, or papillomaviral genes useful for AAV packaging (such as E1A, E1B, E2A, E4 and VA RNA), optionally operably linked to a promoter as one transcriptional unit.

Another aspect of the invention provides a composition or a kit, comprising the recombinant DNA vector of the invention, and a helper plasmid.

In certain embodiments, the helper plasmid comprises a helper virus gene that sufficiently supports AAV packaging.

In certain embodiments, the helper virus gene comprises: (i) an adenovirus gene, optionally an Adenovirus 5 or Adenovirus 2 gene; and/or (ii) a VA nucleic acid encoding functional VA RNA I and II, an E2A gene encoding a functional E2A protein, and an E4 gene encoding a functional E4 protein.

Another aspect of the invention provides a host cell comprising the recombinant DNA vector of the invention, or the composition of the invention.

In certain embodiments, the host cell is a HEK293 cell (such as an Expi293F cell), a VP2 cell, a Vero cell, an HUH7 cell, a HepG2 cell, a HeLa cell, an A549 cell, a BHK cell, or an insect cell (such as Sf9).

In another aspect, the invention provides a method of propagating, amplifying, or producing a recombinant replication-defective AAV viral particle encapsidating the GOI, comprising introducing the recombinant DNA vector (dual transfection vector) (e.g., in a plasmid) of the invention into a host cell prior to, concurrently with, or subsequent to introducing the AAV helper genes (e.g., in a plasmid) into the host cells.

In some embodiments, the dual vector and the AAV helper genes in two separate plasmids are co-transfected into a host cell, thereby producing a recombinant replication-defective AAV viral particle encapsidating the GOI.

In certain embodiments, the method further comprises harvesting the recombinant replication-defective AAV viral particle from the host cell.

In certain embodiments, the host cell is a HEK293 cell (such as an Expi293F cell), a VP2 cell, a Vero cell, an HUH7 cell, a HepG2 cell, a HeLa cell, an A549 cell, a BHK cell, or an insect cell (such as Sf9).

In certain embodiments, the recombinant DNA vector is introduced to the host cell by transient transfection.

With the general principles of the invention set forth herein, the sections below provides further detailed description for various aspects of the invention. It should be understood that any embodiment of the invention can be combined with any one or more additional embodiments of the invention, including those embodiments described in different sections of the application, and those described only in the examples, drawings, or claims.

Furthermore, all disclosures pertaining to “recombinant DNA vector” are also applicable to a “vector” and a “plasmid” described herein.

### 2. Rep and Cap Genes

In certain embodiments, the coding sequence for the AAV Rep and Cap proteins, and the gene of interest (GOI) flanked by AAV ITR sequences, are integrated into a single dual transfection (DT) vector of the invention, e.g., a DT plasmid.

The Rep proteins are important for replication and packaging, while the capsid proteins are assembled to create the protein shell of the AAV, or AAV capsid, which form the outer capsid shell that protects the viral genome, as well as being actively involved in cell binding and internalization. Alternative splicing and alternate initiation codons and promoters result in the generation of four different Rep proteins (Rep78, Rep68, Rep52, and Rep40) from a single open reading frame and the generation of three capsid proteins (VP; VP1/VP2/VP3) from a single open reading frame. While not wishing to be bound by theory, the AAV capsid protein typically comprises a molar ratio of 1:1:10 of VP1:VP2:VP3. As used herein, an “AAV serotype” is defined primarily by the AAV capsid. In some instances, the ITRs are also specifically described by the AAV serotype.

In certain embodiments, the AAV ITR, the AAV Rep, and the AAV Cap are from the same or different AAV serotypes.

In some embodiments, the AAV ITR, the AAV Rep, and the AAV Cap are from AAVPHP.B (PHP.B), AAVPHP.A (PHP.A), AAVG2B-26, AAVG2B-13, AAVTH1.1-32, AAVTH1.1-35, AAVPHP.B2 (PHP.B2), AAVPHP.B3 (PHP.B3), AAVPHP.N/PHP.B-DGT, AAVPHP.B-EST, AAVPHP.B-GGT, AAVPHP.B-ATP, AAVPHP.B-ATT-T, AAVPHP.B-DGT-T, AAVPHP.B-GGT-T, AAVPHP.B-SGS, AAVPHP.B-AQP, AAVPHP.B-QQP, AAVPHP.B-SNP (3), AAVPHP.B-SNP, AAVPHP.B-QGT, AAVPHP.B-NQT, AAVPHP.B-EGS, AAVPHP.B-SGN, AAVPHP.B-EGT, AAVPHP.B-DST, AAVPHP.B-DST, AAVPHP.B-STP, AAVPHP.B-PQP, AAVPHP.B-SQP, AAVPHP.B-QLP, AAVPHP.B-TMP, AAVPHP.B-TTP, AAVPHP.S/G2A12, AAVG2A15/G2A3 (G2A3), AAVG2B4 (G2B4), AAVG2B5 (G2B5), PHP.S, AAV1, AAV2, AAV2G9, AAV3, AAV3a, AAV3b, AAV3-3, AAV4, AAV4-4, AAV5, AAV6, AAV6.1, AAV6.2, AAV6.1.2, AAV7, AAV7.2, AAV8, AAV9, AAV9 K449R, AAV9.11, AAV9.13, AAV9.16, AAV9.24, AAV9.45, AAV9.47, AAV9.61, AAV9.68, AAV9.84, AAV9.9, AAV10, AAV11, AAV12, AAV16.3, AAV24.1, AAV27.3, AAV42.12, AAV42-1b, AAV42-2, AAV42-3a, AAV42-3b, AAV42-4, AAV42-5a, AAV42-5b, AAV42-6b, AAV42-8, AAV42-10, AAV42-11, AAV42-12, AAV42-13, AAV42-15, AAV42-aa, AAV43-1, AAV43-12, AAV43-20, AAV43-21, AAV43-23, AAV43-25, AAV43-5, AAV44.1, AAV44.2, AAV44.5, AAV223.1, AAV223.2, AAV223.4, AAV223.5, AAV223.6, AAV223.7, AAV1-7/rh.48, AAV1-8/rh.49, AAV2-15/rh.62, AAV2-3/rh.61, AAV2-4/rh.50, AAV2-5/rh.51, AAV3.1/hu.6, AAV3.1/hu.9, AAV3-9/rh.52, AAV3-11/rh.53, AAV4-8/r11.64, AAV4-9/rh.54, AAV4-19/rh.55, AAV5-3/rh.57, AAV5-22/rh.58, AAV7.3/hu.7, AAV16.8/hu.10, AAV16.12/hu.11, AAV29.3/bb.1, AAV29.5/bb.2, AAV106.1/hu.37, AAV114.3/hu.40, AAV127.2/hu.41, AAV127.5/hu.42, AAV128.3/hu.44, AAV130.4/hu.48, AAV145.1/hu.53, AAV145.5/hu.54, AAV145.6/hu.55, AAV161.10/hu.60, AAV161.6/hu.61, AAV33.12/hu.17, AAV33.4/hu.15, AAV33.8/hu.16, AAV52/hu.19, AAV52.1/hu.20, AAV58.2/hu.25, AAVA3.3, AAVA3.4, AAVA3.5, AAVA3.7, AAVC1, AAVC2, AAVC5, AAV-DJ, AAV-DJ8, AAVF3, AAVF5, AAVH2, AAVrh.72, AAVhu.8, AAVrh.68, AAVrh.70, AAVpi.1, AAVpi.3, AAVpi.2, AAVrh.60, AAVrh.44, AAVrh.65, AAVrh.55, AAVrh.47, AAVrh.69, AAVrh.45, AAVrh.59, AAVhu.12, AAVH6, AAVLK03, AAVH-1/hu.1, AAVH-5/hu.3, AAVLG-10/rh.40, AAVLG-4/rh.38, AAVLG-9/hu.39, AAVN721-8/rh.43, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAVhu.1, AAVhu.2, AAVhu.3, AAVhu.4, AAVhu.5, AAVhu.6, AAVhu.7, AAVhu.9, AAVhu. 10, AAVhu.11, AAVhu.13, AAVhu.15, AAVhu.16, AAVhu.17, AAVhu.18, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.31, AAVhu.32, AAVhu.34, AAVhu.35, AAVhu.37, AAVhu.39, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.51, AAVhu.52, AAVhu.54, AAVhu.55, AAVhu.56, AAVhu.57, AAVhu.58, AAVhu.60, AAVhu.61, AAVhu.63, AAVhu.64, AAVhu.66, AAVhu.67, AAVhu. 14/9, AAVhu.t 19, AAVrh.2, AAVrh.2R, AAVrh.8, AAVrh.8R, AAVrh.10, AAVrh.12, AAVrh.13, AAVrh.13R, AAVrh.14, AAVrh.17, AAVrh.18, AAVrh.19, AAVrh.20, AAVrh.21, AAVrh.22, AAVrh.23, AAVrh.24, AAVrh.25, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.46, AAVrh.48, AAVrh.48.1, AAVrh.48.1.2, AAVrh.48.2, AAVrh.49, AAVrh.51, AAVrh.52, AAVrh.53, AAVrh.54, AAVrh.56, AAVrh.57, AAVrh.58, AAVrh.61, AAVrh.64, AAVrh.64R1, AAVrh.64R2, AAVrh.67, AAVrh.73, AAVrh.74, AAVrh8R, AAVrh8R A586R mutant, AAVrh8R R533A mutant, AAAV, BAAV, caprine AAV, bovine AAV, AAVhE1.1, AAVhEr1.5, AAVhER1.14, AAVhEr1.8, AAVhEr1.16, AAVhEr1.18, AAVhEr1.35, AAVhEr1.7, AAVhEr1.36, AAVhEr2.29, AAVhEr2.4, AAVhEr2.16, AAVhEr2.30, AAVhEr2.31, AAVhEr2.36, AAVhER1.23, AAVhEr3.1, AAV2.5T, AAV-PAEC, AAV-LK01, AAV-LK02, AAV-LK03, AAV-LK04, AAV-LK05, AAV-LK06, AAV-LK07, AAV-LK08, AAV-LK09, AAV-LK10, AAV-LK11, AAV-LK12, AAV-LK13, AAV-LK14, AAV-LK15, AAV-LK16, AAV-LK17, AAV-LK18, AAV-LK19, AAV-PAEC2, AAV-PAEC4, AAV-PAEC6, AAV-PAEC7, AAV-PAEC8, AAV-PAEC11, AAV-PAEC12, AAV-2-pre-miRNA-101, AAV-8h, AAV-8b, AAV-h, AAV-b, AAV SM 10-2, AAV Shuffle 100-1, AAV Shuffle 100-3, AAV Shuffle 100-7, AAV Shuffle 10-2, AAV Shuffle 10-6, AAV Shuffle 10-8, AAV Shuffle 100-2, AAV SM 10-1, AAV SM 10-8, AAV SM 100-3, AAV SM 100-10, BNP61 AAV, BNP62 AAV, BNP63 AAV, AAVrh.50, AAVrh.43, AAVrh.62, AAVrh.48, AAVhu.19, AAVhu.11, AAVhu.53, AAV4-8/rh.64, AAVLG-9/hu.39, AAV54.5/hu.23, AAV54.2/hu.22, AAV54.7/hu.24, AAV54.1/hu.21, AAV54.4R/hu.27, AAV46.2/hu.28, AAV46.6/hu.29, AAV128.1/hu.43, true type AAV (ttAAV), UPENN AAV 10, Japanese AAV 10 serotypes, AAV CBr-7.1, AAV CBr-7.10, AAV CBr-7.2, AAV CBr-7.3, AAV CBr-7.4, AAV CBr-7.5, AAV CBr-7.7, AAV CBr-7.8, AAV CBr-B7.3, AAV CBr-B7.4, AAV CBr-E1, AAV CBr-E2, AAV CBr-E3, AAV CBr-E4, AAV CBr-E5, AAV CBr-e5, AAV CBr-E6, AAV CBr-E7, AAV CBr-E8, AAV CHt-1, AAV CHt-2, AAV CHt-3, AAV CHt-6.1, AAV CHt-6.10, AAV CHt-6.5, AAV CHt-6.6, AAV CHt-6.7, AAV CHt-6.8, AAV CHt-P1, AAV CHt-P2, AAV CHt-P5, AAV CHt-P6, AAV CHt-P8, AAV CHt-P9, AAV CKd-1, AAV CKd-10, AAV CKd-2, AAV CKd-3, AAV CKd-4, AAV CKd-6, AAV CKd-7, AAV CKd-8, AAV CKd-B1, AAV CKd-B2, AAV CKd-B3, AAV CKd-B4, AAV CKd-B5, AAV CKd-B6, AAV CKd-B7, AAV CKd-B8, AAV CKd-H1, AAV CKd-H2, AAV CKd-H3, AAV CKd-H4, AAV CKd-H5, AAV CKd-H6, AAV CKd-N3, AAV CKd-N4, AAV CKd-N9, AAV CLg-F1, AAV CLg-F2, AAV CLg-F3, AAV CLg-F4, AAV CLg-F5, AAV CLg-F6, AAV CLg-F7, AAV CLg-F8, AAV CLv-1, AAV CLv1-1, AAV Clv1-10, AAV CLv1-2, AAV CLv-12, AAV CLv1-3, AAV CLv-13, AAV CLv1-4, AAV Clv1-7, AAV Clv1-8, AAV Clv1-9, AAV CLv-2, AAV CLv-3, AAV CLv-4, AAV CLv-6, AAV CLv-8, AAV CLv-D1, AAV CLv-D2, AAV CLv-D3, AAV CLv-D4, AAV CLv-D5, AAV CLv-D6, AAV CLv-D7, AAV CLv-D8, AAV CLv-E1, AAV CLv-K1, AAV CLv-K3, AAV CLv-K6, AAV CLv-L4, AAV CLv-L5, AAV CLv-L6, AAV CLv-M1, AAV CLv-M11, AAV CLv-M2, AAV CLv-M5, AAV CLv-M6, AAV CLv-M7, AAV CLv-M8, AAV CLv-M9, AAV CLv-R1, AAV CLv-R2, AAV CLv-R3, AAV CLv-R4, AAV CLv-R5, AAV CLv-R6, AAV CLv-R7, AAV CLv-R8, AAV CLv-R9, AAV CSp-1, AAV CSp-10, AAV CSp-11, AAV CSp-2, AAV CSp-3, AAV CSp-4, AAV CSp-6, AAV CSp-7, AAV CSp-8, AAV CSp-8.10, AAV CSp-8.2, AAV CSp-8.4, AAV CSp-8.5, AAV CSp-8.6, AAV CSp-8.7, AAV CSp-8.8, AAV CSp-8.9, AAV CSp-9, AAV.hu.48R3, AAV.VR-355, AAV3B, AAV4, AAV5, AAVF1/HSC1, AAVF11/HSC11, AAVF12/HSC12, AAVF13/HSC13, AAVF14/HSC14, AAVF15/HSC15, AAVF16/HSC16, AAVF17/HSC17, AAVF2/HSC2, AAVF3/HSC3, AAVF4/HSC4, AAVF5/HSC5, AAVF6/HSC6, AAVF7/HSC7, AAVF8/HSC8, and/or AAVF9/HSC9 and variants thereof.

In certain embodiments, the AAV Cap is a derivative of wild-type AAV9. In certain embodiments, the derivative comprises an insertion of a short peptide (e.g., 3, 4, 5, 6, 7, 8, or 9 residues) in-between residues 588 and 589 of the wild-type AAV9 capsid VP1. In certain embodiments, the insertion comprises, consists essentially of, or consists of RGDLGLS into residues 588 and 589 of wild-type AAV9 Capsid VP1.

In certain embodiments, the rDNA vector/plasmid of the invention comprises a cap gene promoter. The cap gene promoter may be operably linked to a cap gene. In certain embodiments, the cap gene promoter is a native cap gene promoter.

The native cap gene (i.e., the cap gene of a wild type AAV) is operably linked to a p40 promoter, a p5 promoter and a p19 promoter. Thus in one embodiment, the rDNA vector/plasmid of the invention comprises a cap gene promoter such as an AAV p40 promoter, a p5 promoter, and/or a p19 promoter.

The native p40 promoter is contained within the native rep gene. In some embodiments, the p40 promoter has a sequence of or at least 95%, at least 98%, or 99% identical to that of AAV2. In some embodiments, the at least one cap gene promoter is comprised in a promoter region comprising a p40 promoter, a p5 promoter and a p19 promoter.

The native p5 promoter is upstream of the native rep gene. In some embodiments, the p5 promoter has a sequence of or at least 95%, at least 98%, or at least 99% identical to that of AAV2.

In certain embodiments, the wild-type or native p5 promoter is modified to create a modified p5 promoter. In some embodiments, the modified p5 promoter further reduces mis-packaging. In certain embodiments, the modified p5 promoter comprises a REP binding site and a transcription start site-localized Ying-Yang 1 (YY1) binding site. In some embodiments, the modified P5 promoter comprises a REP binding site and a transcription start site-localized Ying-Yang 1 (YY1) binding site with an exogenous spacer sequence inserted between the REP binding site and YY1 binding site; optionally, the spacer is 5 nucleotides to 100 nucleotides in length (e.g., about 5 nts).

Without being bound to a particular theory, insertion of the spacer between the P5 REP binding sequence and the YY1 box at the transcription start site of the P5 promoter may cause or contribute to reduced or eliminated mis-packaging of vector/plasmid DNA upstream of the P5 promoter, while maintaining recombinant AAV titers.

In certain embodiments, the spacer includes an exogenous spacer sequence inserted between the REP binding site and YY1 binding site. In certain embodiments, excluding the exogenous spacer, the modified P5 promoter that is or is at least 80%, 81%, 82%, 83%, 84%, 85%, 86%, 87%, 88%, 89%, 90%, 91%, 92%, 93%, 94%, 95%, 96%, 97%, 98%, 99% or more sequence identity to wild-type P5 promoter, or a length within any range delimited by any pair of the foregoing values, such as between 80% and 99%, between 90% and 99% or between 95% and 99%, for example. In certain embodiments, the modified P5 promoter, into which the exogenous spacer is inserted, has a sequence of nucleotides corresponding to at least about 40, 50, 60, 70, 80, 90, 100, 110, 120, 130, 140, 150, or 160 nucleotides, or a length within any range delimited by any pair of the foregoing values, such as between 40 and 50, between 50 and 160 or between 80 and 130, for example, adjacent (or upstream of) the transcription start site of the large REP proteins (i.e., REP78 and REP68) in an AAV genome, or a sequence having, or having at least, 80%, 81%, 82%, 83%, 84%, 85%, 86%, 87%, 88%, 89%, 90%, 91%, 92%, 93%, 95%, 96%, 97%, 98% or more, including ranges thereof, sequence identity thereto, wherein the promoter drives transcription of the large REP proteins, i.e., REP78 and REP68.

In some examples, the modified P5 promoter has a sequence derived from the sequence of the region adjacent to the transcription start site of REP78 and REP68 in AAV serotype 1 (AAV1), AAV serotype 2 (AAV2), AAV serotype 3 (AAV3), AAV serotype 4 (AAV4), AAV serotype 6 (AAV6}, AAV serotype 7 (AAV7), AAV serotype 8 (AAV8), AAV serotype 9 (AAV9), or any of the following AAV serotype: AAVrh.2, AAVrh.2R, AAVrh.8, AAVrh.8R, AAVrh.10, AAVrh.12, AAVrh.13, AAVrh.13R, AAVrh.14, AAVrh.17, AAVrh.18, AAVrh.19, AAVrh.20, AAVrh.21, AAVrh.22, AAVrh.23, AAVrh.24, AAVrh.25, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.43, AAVrh.46, AAVrh.48, AAVrh.48.1, AAVrh.48.1.2, AAVrh.48.2, AAVrh.49, AAVrh.50, AAVrh.51, AAVrh.52, AAVrh.53, AAVrh.54, AAVrh.56, AAVrh.57, AAVrh.58, AAVrh.61, AAVrh.62, AAVrh.64, AAVrh.64R1, AAVrh.64R2, AAVrh.67, AAVrh.73, AAVrh.74, AAVrh8R, AAVrh8R A586R mutant, AAVrh8R R533A mutant.

In certain embodiments, the modified P5 promoters comprises or consists of a sequence corresponding to the approximately 160 nucleotides adjacent to the transcription start site of REP78 and REP68 in an AAV genome, such as an AAV1, AAV2, AAV3, AAV4, AAV6, AAV7, AAV8, AAV9, AAVrh.2, AAVrh.2R, AAVrh.8, AAVrh.8R, AAVrh.10, AAVrh.12, AAVrh.13, AAVrh.13R, AAVrh.14, AAVrh.17, AAVrh.18, AAVrh.19, AAVrh.20, AAVrh.21, AAVrh.22, AAVrh.23, AAVrh.24, AAVrh.25, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.43, AAVrh.46, AAVrh.48, AAVrh.48.1, AAVrh.48.1.2, AAVrh.48.2, AAVrh.49, AAVrh.50, AAVrh.51, AAVrh.52, AAVrh.53, AAVrh.54, AAVrh.56, AAVrh.57, AAVrh.58, AAVrh.61, AAVrh.62, AAVrh.64, AAVrh.64R1, AAVrh.64R2, AAVrh.67, AAVrh.73, AAVrh.74, AAVrh8R, AAVrh8R A586R mutant, or AAVrh8R R533A mutant genome.

As used herein, “REP binding sequence,” “REP binding site,” or “REP binding element” are used interchangeably herein to refer to an element of the P5 promoter composed of two to four imperfect GAGC repeats that are located just downstream, i.e., 3′, of the TATA box and upstream of the transcription start site of the P5 promoter. Without being bound to a particular theory, Rep-mediated repression is thought to be the result of Rep binding to the REP binding element. In certain embodiments, the P5 REP binding sequence is or comprises GCCCGAGTGAGCACGC.

As used herein, the “Ying-Yang 1 binding site,” “YY1 binding site,” or “YY1 box” refers to a YY1 recognition sequence, which when bound by YY1, supports initiation from the transcription start site. The +1 YY1 binding of the P5 promoter is located at the transcription start site and has the consensus sequence CCAT.

As used herein, the term “exogenous spacer sequence” refers to a sequence that is not native to the P5 promoter.

The exogenous spacer sequence may be inserted anywhere between the REP binding site and YY1 binding site as described herein. In some embodiments, the exogenous spacer sequence is inserted into or replaces one or more of the seven nucleotides located between the REP binding site and YY1 binding site. By way of illustration, the exogenous spacer sequence may be inserted into or replace one or more nucleotides of the sequence AGGGTCT of the AAV8 P5 promoter.

In some embodiments, the total length of the exogenous spacer is 5, 10, 15, 20, 25, 30, 35, 40, 45, 50, 55, 60, 65, 70, 75, 80, 85, 90, 95, 100, 105, 110, 115, 120 or more nucleotides, or a length within any range delimited by any pair of the foregoing values, such as between 5 and 120, between 10 and 100 or between 5 and 100, for example. Ideally, the exogenous spacer is 5 to 100 nucleotides in length. As would be understood by those skilled in the art, the sequence of the spacer is not critical. The sequence of the spacer may be a random, artificial sequence, a sequence derived from AAV or a sequence derived from another source.

The p19 promoter is contained within the native rep gene. In some embodiments, the p19 promoter has a sequence of or at least 95%, at least 98%, or at least 99% identical to that of AAV2.

In some embodiments, the AAV ITR, the AAV Rep and the AAV Cap are from the same AAV. In some other embodiments, the AAV ITR, the AAV Rep and the AAV Cap are from different AAVs. For example, the AAV Rep may be from AAV2, and the AAV Cap may be from AAV9 or a derivative thereof such as the aforementioned wt AAV9 derivatives, and including SLB-101 (see SEQ ID NO: 14 and FIG. 6 of WO2021/072197, incorporated herein by reference).

In certain embodiments, the AAV ITR is AAV2 ITR, the AAV Rep is Rep2 from AAV2, and the AAV Cap is Cap9 from AAV9 (or a derivative thereof).

In certain embodiments, the coding sequence for AAV Rep protein encodes a wild type Rep 40, Rep 52, Rep 68 and/or Rep 78 of an AAV, such as AAV2. In certain embodiments, these Rep proteins are transcribed from one or more of the rep promoters p5, p19 and/or p40.

The rep gene encodes at least one functional Rep proteins (Rep 78, Rep 68, Rep 52 and Rep 40). The gene region is under the control of the p5 and p19 promoters. When the p5 promoter is used, a sequence that encodes Rep 78 and Rep 68 is transcribed. Rep 78 and Rep 68 are two alternative splice variants (Rep 78 comprises an intron that is excised in Rep 68). Similarly, when the p19 promoter is used, a sequence that encodes Rep 52 and Rep 40 is transcribed. Rep 52 and Rep 40 are alternative splice variants (Rep 52 comprises an intron that is excised in Rep 40). The four Rep proteins are known to be involved in replication and packaging of the viral genome, and are thus useful in rAAV production.

In certain embodiments, it is not necessary for all four Rep proteins to be present. In certain embodiments, at least one encoded large Rep protein (Rep 78 or Rep 68) and one encoded small Rep protein (Rep 52 or Rep 40) are present. Rep 78 can be toxic to cells, and Rep 78 does not need to be present in order for AAV replication to take place. Thus, in some embodiments, Rep78 is absent. In some embodiments, the rep gene does not encode or transcribe Rep 78. In certain embodiments, the rep gene encodes/transcribes Rep 68.

In certain embodiments, the rDNA vector/plasmid of the invention comprises at least one rep gene encoding: (a) a functional Rep 52 protein; (b) a functional Rep 40 protein; and/or (c) a functional Rep 68 protein.

As used herein, a “functional” Rep protein is one which allows for production of AAV particles. In particular, Rep 78 or Rep 68 (the large Rep proteins) are believed to be involved in replication of the AAV genome, and Rep 52 and Rep 40 (the small Rep proteins) are believed to be involved in packaging of the AAV genome into a capsid. One of skill in the art can readily determine whether a given Rep protein is functional, such as by determining, e.g., whether the Rep protein supports AAV production using an AAV production assay as described above.

In some embodiments, the at least one rep gene encodes a “functional” Rep protein if the Rep protein supports rAAV production at a level of or at least 25%, of or at least 40%, of or at least 50%, of or at least 70%, of or at least 80%, of or at least 90%, of or at least 95%, or a level within any range delimited by any pair of the foregoing values, such as between 25% and 95%, between 40% and 90% or between 25% and 70%, for example, of the level supported by the wild type Rep protein.

In certain embodiments, the Rep protein is compatible with the ITR(s) surrounding the GOI. Some Rep proteins may only be able to package genomic material (such as an expression cassette) when it is flanked by ITR(s) of the same serotype as the Rep protein. Other Rep proteins are cross compatible, and can package genomic material that is flanked by ITR(s) of a different serotype.

In certain embodiments, the rDNA vector/plasmid of the invention comprises two or more rep genes that are separated on the vector/plasmid. For example, one of the separate rep genes could encode Rep 68 (for example using the p5 promoter or a different promoter situated near the normal position of the p5 promoter in the rep gene), another encoding Rep 40 (for example using the p19 promoter or a different promoter situated near the normal position of the p19 promoter). The one rep gene encoding Rep 40 may also encode Rep 52 since Rep 52 and Rep 40 are alternative splice variants.

In certain embodiments, if two genes encode a Rep 40 protein, one of the two genes that encodes a functional Rep 40 protein may comprise an intron. In one embodiment, both genes that encode a functional Rep 40 protein comprise an intron. In another embodiment, only one of the genes that encodes a functional Rep 40 protein comprises an intron.

For example, to avoid the at least one rep gene encoding Rep 78, the rep gene may be split through partial duplication into two genes. One gene could comprise nucleotides corresponding to the full length native rep gene with the sequence corresponding to the intron removed. Such a gene would encode Rep 68 and Rep 40, but would not encode either Rep 78 or Rep 52, as a portion of each of the Rep 78 and Rep 52 proteins is encoded by the sequence which acts as an intron in the context of rep 40. The second gene could comprise nucleotides corresponding to the region of the native rep gene downstream of the p19 promoter, which would encode Rep 52 (intron spliced in) and Rep 40 (intron spliced out).

In some embodiments, the at least one rep gene does not comprise a functional internal p40 promoter. The native rep/cap gene comprises a p40 promoter (Pereira and Muzyczka, J. Virol. 71:1747-1756, 1997). The p40 promoter drives expression of the cap gene, but is not required for expression of rep genes. A functional p40 promoter is one that is capable to drive expression of the cap gene.

In certain embodiments, the coding sequence for AAV Rep and Cap proteins is under the transcriptional control of a promoter, such as an AAV p5, p19, or p40 promoter, a modified p5 promoter lacking RBE (Rep-Binding Element), or a ubiquitous promoter (such as CMV promoter, EF1a promoter, CAG promoter, CB promoter etc).

In any of the above embodiments, either the rep gene or the cap gene or both may be replaced by a cloning site (e.g., a multicloning site or MCS), in the rDNA vector of the invention, such that a suitable rep and/or cap gene can be cloned/inserted into the respective cloning sites. This will facilitate the swapping of any desired rep or cap genes into the rDNA vector/plasmid of the invention.

### 3. Gene of Interest (GOI) in rAAV and Treatable Diseases

The system and method of the invention can be used to produce recombinant AAV vectors carrying a gene of interest (GOI) flanked by AAV ITR sequences.

In certain embodiments, the rDNA vector (plasmid) of the invention comprises ITR sequences derived from AAV1, AAV2, AAV4 and/or AAV6. In certain embodiments, the ITR sequences are AAV2 ITR sequences.

As used herein, “gene of interest” or GOI or goi generally refers to a nucleic acid or polynucleotide sequence, such as a gene, an open reading frame (ORF), or a coding sequence for protein or RNA, such as non-coding RNA that includes siRNA, miRNA, shRNA, antisense RNA or a precursor thereof. However, in certain circumstances or context, the term GOI also loosely refers to a protein (encoded by the GOI), or a disease or indication that can be remedied by the GOI, or a disease or indication can be (but is not necessarily) caused by loss of function of the GOI.

For example, and merely to illustrate, the gene GALGT2 encodes the protein GalNAc transferase (β-1,4-N-acetylgalactosamine galactosyltransferase), which is an enzyme that transfers a complex sugar molecule onto a few specific proteins, including dystroglycan. Under normal circumstances, GalNAc transferase is found only at the neuromuscular junction (NMJ), where some components of the dystroglycan-associated protein complex are different than elsewhere in muscle. Importantly, at the NMJ, utrophin is present instead of dystrophin. In the mdx mouse model of muscular dystrophy, viral gene transfer of GALGT2 results in expression of GalNAc transferase across the entire muscle membrane, instead of just at the normal expression domain of the NMJ, as well as upregulation of utrophin across the entire muscle fiber. In the mdx mouse, this expression can correct muscle functional deficits to the same degree as does microdystrophin gene expression. Furthermore, overexpression of GALGT2 corrects muscle pathology in mouse models of other muscular dystrophies, including LGMD2A and congenital muscular dystrophy (MDC1A). Thus GALGT2 is a GOI for treating muscular dystrophy such as DMD, BMD, LGMD2A and MDC1A, even though GALGT2 is not necessarily defective per se in the patient in need of treatment.

In another example, Sarcolipin (SLN) inhibits the sarco/endoplasmic reticulum (SR) Ca²⁺ ATPase (SERCA), and is abnormally elevated in the muscle of DMD patients and animal models such as the mdx mouse model of DMD. Reducing SLN levels by AAV9-mediated RNA interference ameliorates dystrophic pathology in the severe dystrophin/utrophin double mutant (mdx:utr^{-/-}) mouse model of DMD, including attenuation of muscle pathology and improvement of diaphragm, skeletal muscle and cardiac function. Thus the coding sequence for SLN RNAi is a GOI that remedies DMD.

Thus the GOI can be a gene (or protein) that, when expressed, replaces a mutated, damaged, or inactive gene or protein. The GOI can be a gene (or protein) that, when expressed, assists an already functioning process that can benefit from further modification for therapy in a disease, disorder, or dysfunction. The GOI can be a gene (or protein) that, when expressed, assists a dysfunctional process that can benefit from further modification for therapy in a disease, disorder, or dysfunction. A GOI nucleic acid sequence can be DNA, RNA, or synthetic nucleic acid molecule. The GOI can be a protein, an enzyme, a structural protein, a functional protein, or an adaptable protein based on cell function(s). The GOI can provide therapeutic benefit or a treatment modality for a disease, disorder, or dysfunction.

In certain embodiments, the recombinant DNA vector of the invention comprises more than one copy of the GOI. In some embodiments, the plasmid of the invention comprises more than one copy of the GOI. In certain embodiments, the multiple GOI copies are the same. In some other embodiments, the multiple GOI are different.

In certain other embodiments, the GOI may be a CRISPR/Cas effector enzyme, such as a Class 2, Type II, IV, V, or VI effector enzyme, including CRISPR-Cas9, Cas 12, Cas 13, etc. In certain embodiments, the GOI may be a TALEN, or other genetic based gene editing protein that functions upon intracellular delivery for their intended activity, such as gene editing or gene knockout in a target cell, tissue, or organism/individual.

In certain embodiments, CRISPR/Cas effector enzyme lacks endonuclease activity (dCas, such as dCas9).

In certain embodiments, the Cas or dCas is further fused to a base editor, such as a cytosine base editor (CBE, e.g., APOBEC, BE1, BE2, BE3, Target-AID base editor, SaBE3, BE3 PAM variants, BE3 editing window variants, AID, CDA1, APOBEC3G, HF-BE3, BE4, BE4max, and AncBE4max), an adenine base editor (ABE, e.g., ABE7.10, ABE 6.3, ABE7.8, ABE7.9, ABEmax, ABE8e (TadA-8e V106W), ABE8 and variants thereof), or a dual base editor (SPACE, A&C-BEmax).

Any and all GOIs as used herein may benefit from codon optimization for enhanced expression and activity via known computer-based algorithms or other codon optimization method, such as manual optimization.

In certain embodiments, the GOI is within a pro-AAV cassette comprising the GOI operably linked to a promoter.

In some embodiments, the promoter operably linked to the GOI is an ubiquitous promoter which drives or promotes expression of the GOI in most tissues. In some embodiments, the promoter is elongation factor 1α-subunit (EF1α) promoter, cytomegalovirus (CMV) immediate-early enhancer promoter, chicken β-actin (CBA) and its derivative CAG promoter, β glucuronidase (GUSB) promoter, or ubiquitin C (UBC) promoter.

In some other embodiments, the promoter operably linked to the GOI is a tissue-specific promoter which can be used to restrict expression to certain cell types such as, but not limited to, muscle specific promoters, B cell promoters, monocyte promoters, leukocyte promoters, macrophage promoters, pancreatic acinar cell promoters, endothelial cell promoters, lung tissue promoters, astrocyte promoters, or nervous system promoters which can be used to restrict expression to neurons, astrocytes, or oligodendrocytes.

Non-limiting examples of muscle-specific promoters include mammalian muscle creatine kinase (MCK) promoter, mammalian desmin (DES) promoter, mammalian troponin I (TNNI2) promoter, and mammalian skeletal alpha-actin (ASKA) promoter.

In some embodiments, the pro-AAV cassette may further comprise an enhancer that promotes the transcription of the GOI from the promoter, a 5′ UTR, a Kozak sequence, a heterologous intron that promotes transcription and/or translation of the GOI, a 3′ untranslated region (UTR), a WPRE sequence, and/or a polyA signal sequence.

In certain embodiments, the pro-AAV cassette comprises a transcription regulatory element comprising the promoter element and/or enhancer element from HLP2, HLP1, LP1, HCR-hAAT, ApoE-hAAT, and/or LSP. These transcription regulatory elements are described in more detail in the following references: HLP2: WO16/075473; HLP1: McIntosh et al., Blood 121(17):3335-44, 2013; LP1: Nathwani et al., Blood 107(7):2653-2661, 2006; HCR-hAAT: Miao et al., Mol Ther. 1:522-532, 2000; ApoE-hAAT: Okuyama et al., Human Gene Therapy 7:637-645, 1996; and LSP: Wang et al., Proc Natl Acad Sci USA. 96(7): 3906-3910, 1999 (all incorporated herein by reference). Each of these transcription regulatory elements may comprise a promoter, an enhancer, and/or optionally other nucleotides.

In certain embodiments, the plasmid of the invention comprises a promoter region comprising one or more promoters, and the promoter region does not comprise dispensable translation initiation codons (e.g., ATG or GTG codons) to prevent unintended/undesirable translation initiation. In certain embodiments, the promoter region comprises p5, p19 and/or p40 promoters, and wherein ATG or GTG codons at one or more positions within these promoters are absent or mutated.

The GOI can be any gene or coding sequence within the packaging capacity of the AAV, e.g., about 4-5 kb, or about 4.7 kb including the ITR sequences, or about 4.4 kb without accounting for the ITR sequences.

The AAV ITR sequences flanking the GOI can be from any AAV ITR, and can be from the same or different AAV serotypes. In certain embodiments, the AAV ITR sequences flanking the GOI are both from AAV1, AAV2, AAV3A, AAV3B, AAV4, AAV5, AAV6, AAV7, AAV8, AAV9, AAV10, AAV11, AAV12, AAV13, AAVrh74, or AAV-DJ.

In some embodiments, the AAV ITR, the AAV Rep and the AAV Cap are from the same AAV. In some other embodiments, the AAV ITR, the AAV Rep and the AAV Cap are from different AAVs.

In certain embodiments, the AAV ITR is AAV2 ITR, the AAV Rep is Rep2 from AAV2, and the AAV Cap is Cap9 from AAV9 (or a derivative thereof).

In certain embodiments, the tropism of the AAV include skeletal muscle (such as AAV1, AAV6, AAV7, AAV8, or AAV9, preferably AAV9, or a derivative thereof, such as SLB-101 (supra)).

In certain aspects, the rAAV that may be produced by using the DT vectors (e.g., DT plasmids) of the invention and the suitable host cells (which can supply any useful helper functions for rAAV production in trans), may encode a gene of interest (GOI) useful for, e.g., gene therapy to treat a disease or condition.

Representative (non-limiting) gene of interest (GOI) may include: a gene responsible for/defective in LGMD2E (limb-girdle muscular dystrophy type 2E), LGMD2D (limb-girdle muscular dystrophy type 2D), LGMD2C (limb-girdle muscular dystrophy type 2C), LGMD2B (limb-girdle muscular dystrophy type 2B), LGMD2L (limb-girdle muscular dystrophy type 2L), LGMD2I (limb-girdle muscular dystrophy type 21), or a gene or coding sequence for NAGLU (α-N-acetylglucosaminidase, for Sanfilippo syndrome or mucopolysaccharidosis type IIIB (MPS IIIB)), sulfamidase or SGSH (for mucopolysaccharidosis type IIIA or MPS IIIA), Factor IX, Factor VIII, Myotubularin 1 (MTM1), Survival of Motor Neuron (SMN, for spinal muscular atrophy or SMA), GalNAc transferase GALGT2, calpain-3 (CAPN-3), acid alpha-glucosidase (GAA, for Pompe disease), alpha-galactosidase A or GLA (for Fabry disease), glucocerebrosidase, dystrophin or microdystrophin.

In certain embodiments, the GOI is a microdystrophin gene.

In certain embodiments, the microdystrophin gene is any such one described in the following patents: U.S. Pat. Nos. 7,906,111; 7,001,761; 7,510,867; 6,869,777; 8,501,920; 7,892,824; WO2016115543; WO 2023/018854, or U.S. Pat. No. 10,166,272, each and every one incorporated herein by reference). In certain embodiments, the GOI is a microdystrophin gene having the nucleotide sequence of SEQ ID NO: 1 of WO 2023/018854 (incorporated herein by reference). In certain embodiments, the microdystrophin gene is capable of being packaged into a rAAV virion, e.g., no more than about 4.7 kb in size.

In certain embodiments, the microdystrophin gene contains within its coding sequence spectrin-like repeats R16 and R17 that are capable of restoring nitric oxide synthase (nNOS) activity to the sarcolemma (such as those described in U.S. Pat. No. 7,892,824).

In certain embodiments, the microdystrophin gene comprises a coding sequence for the R1, R16, R17, R23, and R24 spectrin-like repeats (i.e., SR1, SR16, SR17, SR23, and SR24, respectively) of the full-length dystrophin protein, such as one described in PCT/US2016/013733 (incorporated herein by reference). In certain embodiments, the microdystrophin gene does not encode any other spectrin repeats of the full-length dystrophin protein, other than SR1, SR16, SR17, SR23, and SR24.

Diseases or conditions having a potential to benefit from the rAAV produced by the dual transfection vector of the invention include: Huntington's disease, X-linked myotubular myopathy (XLM™), Acid maltase deficiency (e.g., Pompe disease), Spinal Muscular Atrophy (SMA), Myasthenia Gravis (MG), Amyotrophic lateral sclerosis (ALS), Friedreich's ataxia, Mitochondrial myopathy, Muscular dystrophies (Duchenne's muscular dystrophy, Myotonic dystrophy, Becker muscular dystrophy (BMD), Limb-girdle muscular dystrophy (LGMD), Facioscapulohumeral muscular dystrophy (FSH), Congenital muscular dystrophy (CDM), Oculopharyngeal muscular dystrophy (OPMD), Distal muscular dystrophy, Emery-Dreifuss muscular dystrophy (EDMD), Mucopolysaccharidoses (MPS), Metachromatic leukodystrophy (MLD), Batten Disease, Rett Syndrome, Krabbe Disease, Canavan disease, X-Linked Retinoschisis, Achromatopsia (CNGB3 and CNGA3), X-Linked Retinitis Pigmentosa, Age-Related Macular Degeneration, neovascularized macular degeneration, Pompe, Fabry's disease, MPS I, II, IIIA, IIIB, Gaucher's disease, Dannon Disease, A1At Deficiency, Friedreich ataxia, Wilson's Disease, Batten Disease (CLN1, CLN3, CLN6, CLN8), Wolman Disease, Tay-Sachs, Niemann-Lick Type C, CDKL5 deficiency Disorder, B-thalassemia, Sickle cell disease.

In certain embodiments, diseases or conditions having a potential to benefit from the rAAV produced by the dual transfection vector of the invention may include: Becker muscular dystrophy (BMD), Congenital muscular dystrophies (CMD), Bethlem CMD, Fukuyama CMD, Muscle-eye-brain diseases (MEBs), Rigid spine syndromes, Ullrich CMD, Walker-Warburg syndromes (WWS), Duchenne muscular dystrophy (DMD), Emery-Dreifuss muscular dystrophy (EDMD), Facioscapulohumeral muscular dystrophy (FSHD), Limb-girdle muscular dystrophies (LGMD), Myotonic dystrophy (DM), Oculopharyngeal muscular dystrophy (OPMD), Motor neuron diseases including ALS (amyotrophic lateral sclerosis), Spinal-bulbar muscular atrophy (SBMA), Spinal muscular atrophy (SMA).

In certain embodiments, diseases or conditions having a potential to benefit from the rAAV produced by the dual transfection vector of the invention may include ion channel diseases, which are typically marked by muscular weakness, absent muscle tone, or episodic muscle paralysis. They include Andersen-Tawil syndrome, Hyperkalemic periodic paralysis, Hypokalemic periodic paralysis, Myotonia congenita, Becker myotonia, Thomsen myotonia, Paramyotonia congenita, Potassium-aggravated myotonia.

In certain embodiments, diseases or conditions having a potential to benefit from the rAAV produced by the dual transfection vector of the invention may include mitochondrial diseases, which occur when structures that produce energy for a cell malfunction. Such diseases include: Friedreich's ataxia (FA), Mitochondrial myopathies, Kearns-Sayre syndrome (KSS), Leigh syndrome (subacute necrotizing encephalomyopathy), Mitochondrial DNA depletion syndromes, Mitochondrial encephalomyopathy, lactic acidosis and stroke-like episodes (MELAS), Mitochondrial neurogastrointestinal encephalomyopathy (MNGIE), Myoclonus epilepsy with ragged red fibers (MERRF), Neuropathy, ataxia and retinitis pigmentosa (NARP), Pearson syndrome, Progressive external opthalmoplegia (PEO).

In certain embodiments, diseases or conditions having a potential to benefit from the rAAV produced by the dual transfection vector of the invention may include myopathies, which is a disease of muscle in which the muscle fibers do not function properly, resulting in muscular weakness. Myopathies include: Cap myopathies, Centronuclear myopathies, Congenital myopathies with fiber type disproportion, Core myopathies, Central core disease, Multiminicore myopathies, Myosin storage myopathies, Myotubular myopathy, Nemaline myopathies, Distal myopathies, GNE myopathy/Nonaka myopathy/hereditary inclusion-body myopathy (HIBM), Laing distal myopathy, Markesberg-Griggs late-onset distal myopathy, Miyoshi myopathy, Udd myopathy/tibial muscular dystrophy, Vocal cord and pharyngeal distal myopathy, Welander distal myopathy, Endocrine myopathies, Hyperthyroid myopathy, Hypothyroid myopathy, Inflammatory myopathies, Dermatomyositis, Inclusion-body myositis, Polymyositis, Metabolic myopathies, Acid maltase deficiency (AMD, Pompe disease), Carnitine deficiency, Carnitine palmityl transferase deficiency, Debrancher enzyme deficiency (Cori disease, Forbes disease), Lactate dehydrogenase deficiency, Myoadenylate deaminase deficiency, Phosphofructokinase deficiency (Tarui disease), Phosphoglycerate kinase deficiency, Phosphoglycerate mutase deficiency, Phosphorylase deficiency (McArdle disease), Myofibrillar myopathies (MFM), Scapuloperoneal myopathy.

In certain embodiments, diseases or conditions having a potential to benefit from the rAAV produced by the dual transfection vector of the invention may include neuromuscular junction diseases, which result from the destruction, malfunction or absence of one or more key proteins involved in the transmission of signals between muscles and nerves. Such diseases include: Congenital myasthenic syndromes (CMS), Lambert-Eaton myasthenic syndrome (LEMS), Myasthenia gravis (MG).

In certain embodiments, diseases or conditions having a potential to benefit from the rAAV produced by the dual transfection vector of the invention may include peripheral nerve diseases, in which the motor and sensory nerves that connect the brain and spinal cord to the rest of the body are affected, causing impaired sensations, movement or other functions. Such diseases include: Charcot-Marie-Tooth disease (CMT), Giant axonal neuropathy (GAN), muscle wasting in cachexia and aging.

In certain embodiments, the GOI coding sequence comprises a polyA signal sequence or polyadenylation site.

In certain embodiments, the polyadenylation site is a bovine growth hormone (bGH) polyadenylation site.

In certain embodiments, the polyadenylation site or poly(A) signal sequence is from other suitable sources, e.g., synthetic sequences or sequences from other eukaryotic genes or viruses.

In certain embodiments, the GOI coding sequence is partially or fully codon-optimized for expression in a mammalian host cell. For example, the most 3′ 300-350 nucleotides of the coding sequence may be codon-optimized for expression in the mammalian host cell.

### 4. Composition or Kits of Two-Vector (e.g., Two-Plasmid) System

One aspect of the invention provides a composition or a kit, comprising a two-vector (e.g., two-plasmid) system that comprises, consists essentially of, or consists of any one of the dual recombinant DNA vector (dual transfection vector or “DT vector”) of the invention, and a helper plasmid sufficient to support AAV packaging in the presence of the DT vector of the invention.

The two-vector (two-plasmid) system in the composition or kit of the invention is useful for producing rAAV. The two-vector (two-plasmid) system in the composition or kit of the invention is suitable for use in producing rAAV. The two-vector (two-plasmid) system in the composition or kit of the invention is for producing rAAV. The two-vector (two-plasmid) system in the composition or kit of the invention is for producing rAAV suitable for use in gene therapy. The two-vector (two-plasmid) system in the composition or kit of the invention is for producing rAAV for use in gene therapy.

The phrase “two-vector (two-plasmid) system” refers to a system that comprises two vectors (e.g., plasmids), and can be used together, without the need for additional plasmids to produce rAAV (i.e., they in combination are sufficient for AAV production). In certain embodiments, the two-vector (plasmid) system can be used to produce rAAV without the need for helper virus such as adenovirus. In certain embodiments, the two-vector (plasmid) system can be used to produce rAAV without the need for genetic material originating from a host cell, optionally with the exception of a gene encoding E1 A/B. However, the vector system in the composition/kit of the invention may comprise additional non-plasmid components. Optionally, the composition or kit of the invention comprising the two-vector (plasmid) system of the invention does not comprise a helper virus.

In certain embodiments, the two-vector (plasmid) system of the invention comprises all the necessary genetic information for the production of rAAV. For example, the two-vector (plasmid) system of the invention may comprise at least one rep gene, at least one cap gene and at least one helper gene. In certain embodiments, the two-plasmid system of the invention comprises all the necessary genetic information useful for the production of rAAV suitable for use in gene therapy. For example, the two-plasmid system of the invention may comprise at least one rep gene, at least one cap gene, at least one helper gene and an expression cassette comprising a transgene operably linked to at least one regulatory control element.

One of the vectors/plasmids in the composition or kit of the invention is the vector/plasmid comprising the AAV Rep, Cap and GOI (flanked by AAV ITR sequences). The other vector/plasmid in the composition or kit of the invention is the helper plasmid/vector.

In certain embodiments, the helper plasmid/vector may comprise at least one helper virus gene. AAV is only able to propagate in the presence of a helper virus. Examples of helper viruses include adenoviruses, and herpes viruses.

In certain embodiments, the helper plasmids of the invention comprise sufficient helper genes to allow for AAV replication and packaging. In certain embodiments, the helper plasmid or vector that can be used with the rDNA vector of the invention provides all the helper genes/functions for AAV packaging (except for the ITR sequences flanking the GOI).

For example, for host cells expressing the adenoviral E1A/B genes, such as the HEK293T cells, the remaining adenoviral helper genes encode E4, E2A and VA RNA I and II. These helper genes can be included in the helper plasmid or vector in any order or orientation, so long as all these genes can be transcribed and expressed to facilitate AAV packaging.

Whether a helper plasmid comprises sufficient helper genes to facilitate AAV production can be assessed using any art recognized AAV production assay to show sufficient titer of AAV so produced.

In one embodiment, the helper gene products will be considered (sufficient) to facilitate AAV production if they support rAAV production at a level of or at least 25%, of or at least 40%, of or at least 50%, of or at least 60%, of or at least 70%, of or at least 80%, of or at least 90%, of or at least 95%, or a level within any range delimited by any pair of the foregoing values, such as between 50% and 95%, between 60% and 80%, or between 80% and 95%, for example, of the level supported by adenoviral helper genes encoding E4, E2A and VA RNA I and II. In certain embodiments, if the yield of rAAV produced is or is at least 25%, or is at least 40%, or is at least 50%, or is at least 60%, or is at least 70%, or is at least 80%, or is at least 90%, or is at least 95%, or a level within any range delimited by any pair of the foregoing values, such as between 50% and 95%, between 60% and 80%, or between 80% and 95%, or a level within any range delimited by any pair of the foregoing values, such as between 50% and 95%, between 60% and 80%, or between 80% and 95%, for example, of the yield of rAAV produced using the reference two-plasmid system in which one is a helper plasmid comprising adenoviral helper genes encoding E4, E2A and VA RNA I and II.

Since the helper plasmids encode the genes that allow for efficient AAV production, the addition of a helper virus is not required (i.e., the composition or kit of the invention comprising the two vector/plasmid of the invention is sufficient to support AAV production and packaging).

In certain embodiments, the at least one helper virus gene comprises an adenovirus gene. Adenovirus is a virus which is known to aid propagation of AAV (Xiao et al., J. Virol, 72:2224-2232, 1998). In certain embodiments, the at least one helper virus gene comprises an Adenovirus 5 gene or an Adenovirus 2 gene. In certain embodiments, the helper genes of adenoviruses encode EIA, E1B, E4, E2A and VA RNA I and II.

E1A is encoded by nucleotides 560-1545 of the Adenovirus 5 genome. The E1A may optionally lack the non-essential intron at nucleotides 1113-1228.

E1B is actually two proteins E1B 19K and E1B 55K, which work together to block apoptosis in adenovirus-infected cells. E1B is encoded by nucleotides 1714-2244 (E1B 19K), and by nucleotides 2019-3509 (E1B 55 K) of the Adenovirus 5 genome.

E4 is encoded by a number of different open reading frames (ORFs) of the Adenovirus 5 genome. E4 ORF 6/7 is encoded by nucleotides 32914-34077, which may optionally lack the intron between nucleotides 33193 and 33903. E4 34K is encoded by nucleotides 33193-34077 of the Adenovirus 5 genome. E4 ORF 4 is encoded by nucleotides 33998-34342 of the Adenovirus 5 genome. E4 ORF 3 is encoded by nucleotides 34353-34703 of the Adenovirus 5 genome. E4 ORF B is encoded by nucleotides 34700 to 35092 of the Adenovirus 5 genome. E4 ORF 1 is encoded by nucleotides 35140-35526 of the Adenovirus 5 genome. A functional E4 protein may only comprise amino acids encoded by ORFs 6 and 7, as only the amino acids encoded by ORFs 6 and 7 are mandatory for activity. In certain embodiments, the functional E4 protein comprises a polypeptide sequence encoded by all or a significant portion of ORFs 6 and 7. Optionally, the functional E4 protein does not comprise polypeptide sequence encoded by all or a portion of ORFs 1~4 and 34K.

In certain embodiments, the functional E4 protein comprises amino acids encoded by ORFs 1-7, as the amino acids encoded by ORFs 1-3 and 34K of E4, though not required, do improve the activity of the E4 protein.

The E2 (E2A) gene is encoded by nucleotides 22443-24032 of the Adenovirus 5 genome.

The VA RNA I and II is encoded by nucleotides 10589-11044 of the Adenovirus 5 genome.

E1B and E4 are believed to enhance AAV mRNA accumulation, and E2A and VA RNA I and II are believed to enhance AAV mRNA splicing and translation. E1B, E4 and E2A are proteins encoded by genes present in the adenovirus genome, whereas the VA nucleic acid encodes two RNA transcripts known as VA RNA I and VA RNA II. The transcripts themselves are functional in the cell, and are never translated into amino acid sequences. It will be appreciated, therefore, that the VA nucleic acid does not encode a protein, but does “encode” or “correspond to” an RNA, i.e., whilst the term “encode” is used the VA nucleic acid is a non-translated nucleic acid sequence.

Of the five adenovirus genes, in some embodiment, it is possible not to include E1A or E1B in the helper plasmid, as some host cell lines (such as HEK293 cells) express one or more of E1A or E1B constitutively. In certain embodiments, the helper plasmid does not comprise a gene encoding a functional adenoviral E1A/B protein.

In one embodiment, the at least one helper virus gene comprises: (a) a VA (viral associated) nucleic acid encoding functional VA RNA I and II; (b) an E2A gene encoding a functional E2A protein; and/or (c) n E4 gene encoding a functional E4 protein.

In certain embodiments, the at least one helper virus gene comprises a VA nucleic acid (such as VA RNA I and/or II), an E2A gene and an E4 gene.

A “functional” VA RNA I and II, E2A protein or E4 protein is able to facilitate production of AAV.

One of skill in the art can determine whether a given VA RNA I and II, E2A protein or E4 protein is functional by determining, for example, whether the VA RNA I and II, E2A protein or E4 protein supports AAV production using an AAV production assay as described above. In some embodiments, the VA RNA I and II, E2A protein or E4 protein is considered to be functional if it supports rAAV production at a level of or at least 25%, of or at least 40%, of or at least 50%, of or at least 70%, of or at least 80%, of or at least 90%, of or at least 95%, or a level within any range delimited by any pair of the foregoing values, such as between 50% and 95%, between 60% and 80%, or between 80% and 95%, for example, of the level supported by the wild type (for example as found in native Adenovirus 5) VA RNA I and II, E2A protein or E4 protein. In certain embodiments, the E4 protein will be considered to be “functional” if it supports rAAV production at a level of or at least 70%, of or at least 80%, of or at least 90%, of or at least 95% of the level supported by the wild type E4 protein.

In certain embodiments, the E4 gene is not located between the VA nucleic acid and the E2A gene, i.e., the sequence of the plasmid is such that E4 gene sequence does not appear in the plasmid between the VA nucleic acid sequence and the E2A gene sequence. In certain other embodiments, the E2A gene is located between the VA nucleic acid and the E4 gene.

Any of the genes on the helper plasmid can take any orientation without regard to the other genes.

In certain embodiments, the VA nucleic acid has an activity level which has or has at least 75%, has or has at least 80%, has or has at least 90%, has or has at least 95%, or between 95% and 100% of the activity of a wild type VA nucleic acid from Adenovirus 5. The activity level of the VA nucleic acid can be determined by, for example, measuring rAAV yield, for example using the AAV production assay described above.

Expression of the E4 gene is driven by a promoter (designated “E4 promoter”). A fully active promoter comprises nucleotides corresponding to 35793-35848 of the Adenovirus 5 genome. In certain embodiments, the E4 gene is operably linked to an E4 promoter that has at least 50%, at least 70%, or at least 90% of the activity of a wild type promoter from Adenovirus 5. The activity of the E4 promoter may be determined by testing its ability to drive expression of a protein. In one embodiment, the E4 promoter has or has at least 25%, has or has at least 40%, has or has at least 50%, has or has at least 70%, has or has at least 90% of the activity of a wild type E4 promoter from Adenovirus 5 if it supports rAAV production at a level of or at least 25%, of or at least 40%, of or at least 50%, of or at least 60%, of or at least 70%, of or at least 90% of the level supported by a wild type E4 gene promoter, i.e., if the yield of rAAV produced is or is at least 50%, or is at least 70%, is or is at least 90% of the yield of rAAV produced using the reference two-plasmid system comprising wild type helper genes with wild-type promoters. Preferably, the E4 promoter will be considered to facilitate AAV production if it supports rAAV production at a level of or at least 70%, of or at least 80%, of or at least 90%, of or at least 95% of the yield of rAAV produced using the reference two-plasmid system comprising wild type helper genes with wild-type promoters.

### 5. Host Cells

One aspect of the invention comprises a host cell comprising the dual recombinant DNA vector (dual transfection vector) of the invention.

In general, a host cell of the invention is capable or suitable for the production of rAAV. The host cell is typically derived from a eukaryotic cell line, such as a vertebrate cell line, including a mammalian cell line (e.g., a human cell line).

In certain embodiments, the host cell is a cell selected from the group consisting of a HEK293T cell, a HEK293 cell, a HEK293EBNA cell, a CAP cell, a CAP-T cell, an AGE1.CR cell, a PerC6 cell, a C139 cell, an EB66 cell, a BHK cell, a COS cell, a Vero cell, a Hela cell, and an A549 cell.

In certain embodiments, the host cell is selected from the group consisting of a HEK293T cell, a HEK293 cell, a HEK293EBNA cell, a CAP cell, a CAP-T cell, an AGE1.CR cell, a PerC6 cell, a C139 cell, and an EB66 cell.

In certain embodiments, the host cell is selected from the group consisting of a HEK293T cell, a HEK293 cell, and a HEK293EBNA cell.

In certain embodiments, the host cell is a HEK293T cell.

In certain embodiments, the host cell is a cell that expresses a functional adenoviral E1A/B protein. For example, the host cell may comprise a chromosome comprising a gene encoding a functional adenoviral E1 A/B protein.

The host cell is considered suitable or capable of producing recombinant AAV if it supports AAV production at a level of or at least 30%, of or at least 40%, of or at least 50%, of or at least 70%, of or at least 80%, of or at least 90%, of or at least 95% of the level supported by HEK293T cells. In this case, the test two-vector/plasmid system of the invention can be introduced/transfected into the host cell whose suitability for the production of recombinant AAV is to be tested, and the reference two-vector/plasmid system is be introduced/transfected into HEK293T cells.

In certain embodiments, the dual transfection vector and/or the helper plasmid (or equivalent function DNA coding sequences) is stably integrated into the host cell genome.

In certain embodiments, the host cell is derived from a vertebrate, such as human, monkey, bovine, porcine, equine and other equids, canine, feline, ovine, goat, murine, rat, rabbit, mink, opossum, camel and other cameloids, chicken and other avian, armadillo, frog, or reptile, or derived from an insect cell.

In certain embodiments, the host cell is a cell line suitable for AAV packaging, such as Expi cells, HEK293 cells (or a derivative thereof such as HEK293T cells).

In certain embodiments, the host cell is a HEK293 cell (such as an Expi293F cell), a HeLa cell, an A549 cell, a BHK cell, or an insect cell (such as Sf9).

In certain embodiments, the host cell is HEK293 (human embryonic kidney), which can be grown using standard tissue culture media such as DMEM complemented with L-Gln, 5-10% fetal bovine serum (FBS), and 1% penicillin-streptomycin.

In some embodiments, the HEK293 cells are grown on a solid support, including tissue culture plates, dishes, flasks, and bottles. For growing adherent HEK293 cells, the percentage of FBS can be reduced during rAAV production in order to limit contamination by animal-derived components.

In some embodiments, the HEK293 cells are adapted to grow in suspension.

In certain embodiments, the host cell is a Vero cell, such as a Vero75.4 or V75 cell described herein. Such cells may grow on a solid support, including tissue culture plates, dishes, flasks, bottles, and microcarrier that allows the adherent Vero cells to grow in suspension-like conditions.

In certain embodiments, the host cell is a BHK (baby hamster kidney) cell, such as BHK21 or sBHK27. In certain embodiments, the BHK cells are adapted to grow in serum-free suspension.

In certain embodiments, the host cell is a HEK293 cell. In certain embodiments, the HEK293 cell is adapted for growth in serum-free media (such as F17 or Expi293 media) and in suspension, thus is amenable for large scale growth in a bioreactor. See, for example, Grieger et al. (Mol. Ther. 24:287-297, 2016, incorporated herein by reference). In certain embodiments, the HEK293 cell is a HEK293T cell which expresses SV40 T antigen (the temperature sensitive allele tsA1609) and the neomycin/geneticin-resistance gene.

In certain embodiments, for production of rAAV particles, the host cell comprises helper virus proteins useful (e.g., required) for AAV packaging.

In some embodiments, the coding sequences of the helper virus proteins are introduced into the host cell in a plasmid through transfection. In some other embodiments, the coding sequences of the helper virus proteins are integrated into the host cell genome.

In certain embodiments, the host cell of the invention may be adapted for use in producing recombinant AAV vectors encoding a gene of interest (GOI), which may be used in gene therapy. See the section entitled “Recombinant AAV Production” below. In such embodiments, one or more rAAV production cell lines may be infected by the DT vectors of the invention, such as vectors or plasmids encoding AAV Rep and Cap proteins.

In certain embodiments, such producer/host cell line for rAAV production is a HeLa- or A549-derived cell line transfected with the DT vector/plasmid of the invention, optionally containing a drug selection marker.

In certain embodiments, such producer cell line for rAAV production is a Vero cell.

In certain embodiments, such producer cell line for rAAV production is a BHK cell.

In certain embodiments, such producer cell line for rAAV production is a HEK293 cell.

In certain embodiments, such producer/host cell line for rAAV production comprises a DT vector/vector of the invention comprising the GOI flanked by the AAV ITR sequences. The GOI can be any one of the GOI described herein useful for gene therapy, such as a dystrophin minigene or a microdystrophin gene described in U.S. Pat. Nos. 7,906,111; 7,001,761; 7,510,867; 6,869,777; 8,501,920; 7,892,824; WO2020/086844; or U.S. Pat. No. 10,166,272, WO 2023/018854, or in PCT/US2016/013733 (all incorporated herein by reference).

For example, PCT/US2016/013733 (WO2016/115543A2) provides a micro-dystrophin gene operatively connected to a regulatory cassette, wherein the micro-dystrophin gene encodes a protein comprising: an amino-terminal actin-binding domain; a β-dystroglycan binding domain; and a spectrin-like repeat domain, comprising at least four spectrin-like repeats, wherein two of the at least four spectrin-like repeats comprise a neuronal nitric oxide synthase binding domain. In certain embodiments, the at least four spectrin-like repeats include spectrin-like repeat 1 (SR1), spectrin-like repeat 16 (SR16), spectrin-like repeat 17 (SR17), and spectrin-like repeat 24 (SR24). In certain embodiments, the protein encoded by the micro-dystrophin gene further comprises at least a portion of a hinge domain, such as at least one of a Hinge 1 domain, a Hinge 2 domain, a Hinge 3 domain, a Hinge 4 domain, and a hinge-like domain. In certain embodiments, the micro-dystrophin gene comprises, in N- to C-terminal order: a Hinge 1 domain (H1); a spectrin-like repeat 1 (SR1); a spectrin-like repeat 16 (SR16); a spectrin-like repeat 17 (SR17); a spectrin-like repeat 24 (SR24); and a Hinge 4 domain (H4). In certain embodiments, H1 is directly coupled to the SR1. In certain embodiments, SR 1 is directly coupled to SR16. In certain embodiments, SR16 is directly coupled to SR17. In certain embodiments, SR 17 is directly coupled to SR24. In certain embodiments, SR24 is directly coupled to the H4. In certain embodiments, the protein encoded by the micro-dystrophin gene further comprises between SR1 and SR16, in N- to C-terminal order, a spectrin-like repeat 2 (SR2) and a spectrin-like repeat 3 (SR3). In certain embodiments, SR1 is directly coupled to SR2 and SR2 is further coupled to SR3. In certain embodiments, H1 is directly coupled to SR1, SR1 is directly coupled to SR16, SR16 is directly coupled to SR17, SR17 is directly coupled to SR23, SR23 is directly coupled to SR24, and SR24 is directly coupled to H4.

In certain embodiments, the regulatory cassette is selected from the group consisting of a CK8 promoter and a cardiac troponin T (cTnT) promoter. In certain embodiments, the protein encoded by the micro-dystrophin gene has between five spectrin-like repeats and eight spectrin-like repeats. In certain embodiments, the protein encoded by the micro-dystrophin gene has at least 80% or 90% sequence identity to the amino acid sequence of SEQ ID NO: 4 or 5 in WO2016/115543A2 (incorporated herein by reference).

In certain embodiments, the GOI is a microdystrophin gene having the nucleotide sequence of SEQ ID NO: 1 of WO 2023/018854 (incorporated herein by reference).

### 6. Recombinant AAV Production

The recombinant DNA vector (dual transfection vector) of the present invention, as well as the production cell lines, can be used for large scale production of recombinant AAV vectors (rAAV) useful for gene therapy. In certain embodiments, the system and the associated method of use, can be used for propagating/amplifying/producing of AAV viral particles comprising the ITR-flanked GOI.

Recombinant replication-defective AAV vectors, which can be produced with the present recombinant DNA vectors (dual transfection vectors) and production cell lines, typically comprise a gene of interest (GOI) and expression regulators (such as promoters for the GOI) in lieu of the wild-type AAV virus rep and cap open reading frames (ORFs). The AAV rep and cap ORFs, optionally their native promoters p5, p19, and p40 are supplied by the dual transfection vector of the invention, and other helper functions useful for AAV packaging are supplied in another construct or production cell line. The rep ORF encodes four nonstructural Rep proteins involved in the AAV viral life cycle, and the cap ORF encodes the three structural proteins (i.e., VP1, VP2, and VP3) that form the icosahedral AAV capsid. Typically, the only AAV viral sequences that are retained in the rAAV vector genome are the inverted terminal repeats (ITRs)—the minimal cis-acting elements for AAV DNA replication and packaging.

In certain aspects, the method of propagating/amplifying/producing a replication-defective AAV particle encapsidating the GOI comprises introducing the recombinant DNA of the invention into a host cell, prior to, concurrently with, or subsequent to introducing the AAV helper genes for virus packaging.

In some embodiments, the recombinant DNA vector is introduced to the host cell by transient transfection.

The method for producing a replication-defective AAV particle comprises the steps of simultaneously co-transfecting mammalian cells, such as, but not limited to HEK293 cells, with the recombinant DNA of the invention and a construct comprising the AAV helper genes.

In some embodiments, the AAV helper genes are introduced into the host cell through transfection, e.g., transient transfection. In some other embodiments, the AAV helper genes are integrated into the host cell genome prior to introduction of the dual transfection vector of the invention to the host cell.

In certain embodiments, the host cell is derived from a vertebrate, such as human, monkey, bovine, porcine, equine and other equids, canine, feline, ovine, goat, murine, rat, rabbit, mink, opossum, camel and other cameloids, chicken and other avian, armadillo, frog, or reptile, or derived from an insect cell. Human cells include BHK cells, Vero cells, HEK293 cells, etc.

In certain embodiments, the host cell is a HEK293 cell (such as an Expi293F cell or VP2 cell), a HeLa cell, an A549 cell, a BHK cell, or an insect cell (such as Sf9).

In certain embodiments, the host cell is HEK293 (human embryonic kidney), which can be grown using standard tissue culture media such as DMEM complemented with L-Gln, 5-10% fetal bovine serum (FBS), and 1% penicillin-streptomycin.

In some embodiments, the HEK293 cells are grown on a solid support, including tissue culture plates, dishes, flasks, and bottles. For growing adherent HEK293 cells, the percentage of FBS can be reduced during rAAV production in order to limit contamination by animal-derived components.

In some embodiments, the HEK293 cells are adapted to grow in suspension.

In certain embodiments, the HEK293 cell is adapted for growth in serum-free media (such as F17 or Expi293 media) and in suspension, thus is amenable for large scale growth in a bioreactor. See, for example, Grieger et al. (Mol. Ther. 24:287-297, 2016, incorporated herein by reference).

In certain embodiments, the HEK293 cell is a HEK293T cell which expresses SV40 T antigen (the temperature sensitive allele tsA1609) and the neomycin/geneticin-resistance gene.

In certain embodiments, the host cell is a Vero cell, such as a Vero75.4 or V75 cell described herein. Such cells may grow on a solid support, including tissue culture plates, dishes, flasks, bottles, and microcarrier that allows the adherent Vero cells to grow in suspension-like conditions.

In certain embodiments, the host cell is a BHK (baby hamster kidney) cell, such as BHK21 or sBHK27. In certain embodiments, the BHK cells are adapted to grow in serum-free suspension.

In some embodiments, the method further comprises harvesting the replication-defective AAV particle comprising the GOI. In some embodiments, the method comprises purifying the recombinant replication-defective AAV particle.

The gene of interest (GOI) may include genes useful for gene therapy in treating certain diseases or conditions. Representative (non-limiting) GOI may include a gene responsible for/defective in LGMD2E (limb-girdle muscular dystrophy type 2E), LGMD2D (limb-girdle muscular dystrophy type 2D), LGMD2C (limb-girdle muscular dystrophy type 2C), LGMD2B (limb-girdle muscular dystrophy type 2B), LGMD2L (limb-girdle muscular dystrophy type 2L), LGMD2I (limb-girdle muscular dystrophy type 21), or a gene or coding sequence for NAGLU (α-N-acetylglucosaminidase, for Sanfilippo syndrome or mucopolysaccharidosis type IIIB (MPS IIIB)), sulfamidase or SGSH (for mucopolysaccharidosis type IIIA or MPS IIIA), Factor IX, Factor VIII, Myotubularin 1 (MTM1), Survival of Motor Neuron (SMN, for spinal muscular atrophy or SMA), GalNAc transferase GALGT2, calpain-3 (CAPN-3), acid alpha-glucosidase (GAA, for Pompe disease), alpha-galactosidase A or GLA (for Fabry disease), glucocerebrosidase, dystrophin or microdystrophin.

Suitable microdystrophin genes include those described in the following patents: U.S. Pat. Nos. 7,906,111; 7,001,761; 7,510,867; 6,869,777; 8,501,920; 7,892,824; WO2016115543; WO 2023/018854; U.S. Pat. No. 10,166,272; WO2020/086844 (all incorporated herein by reference). In certain embodiments, the GOI is a microdystrophin gene having the nucleotide sequence of SEQ ID NO: 1 of WO 2023/018854 (incorporated herein by reference).

Diseases or conditions having a potential to benefit from the rAAV produced by the DT vector of the invention-based system include: Huntington's disease, X-linked myotubular myopathy (XLMTM), Acid maltase deficiency (e.g., Pompe disease), Spinal Muscular Atrophy (SMA), Myasthenia Gravis (MG), Amyotrophic lateral sclerosis (ALS), Friedreich's ataxia, Mitochondrial myopathy, Muscular dystrophies (Duchenne's muscular dystrophy, Myotonic dystrophy, Becker muscular dystrophy (BMD), Limb-girdle muscular dystrophy (LGMD), Facioscapulohumeral muscular dystrophy (FSH), Congenital muscular dystrophy (CDM), Oculopharyngeal muscular dystrophy (OPMD), Distal muscular dystrophy, Emery-Dreifuss muscular dystrophy (EDMD), Mucopolysaccharidoses (MPS), Metachromatic leukodystrophy (MLD), Batten Disease, Rett Syndrome, Krabbe Disease, Canavan disease, X-Linked Retinoschisis, Achromatopsia (CNGB3 and CNGA3), X-Linked Retinitis Pigmentosa, Age-Related Macular Degeneration, neovascularized macular degeneration, Pompe, Fabry's disease, MPS I, II, IIIA, IIIB, Gaucher's disease, Dannon Disease, A1At Deficiency, Friedreich ataxia, Wilson's Disease, Batten Disease (CLN1, CLN3, CLN6, CLN8), Wolman Disease, Tay-Sachs, Niemann-Lick Type C, CDKL5 deficiency Disorder, B-thalassemia, Sickle cell disease, etc.

Being a naturally replication-defective human parvovirus, wild-type AAV integrates its genome site-specifically within the host cell chromosome in the absence of helper assistance for its replication, where it persists indefinitely unless rescued via cellular infection with a helper virus. The introduction of a helper virus into the host cell triggers AAV replication and the generation of progeny virions. In the case of rAAV virions useful for gene therapy, introduction of the helper virus function into a suitable host cell triggers the packaging of the GOI in the rAAV virions, when the requisite rep and cap coding sequences are also supplied in the same system.

In other words, production of recombinant AAV relies on (1) the presence of the AAV rep and cap coding sequences, and (2) the helper virus functions.

In certain embodiments, the tropism of the AAV include serotypes such as AAV1, AAV2, AAV6, AAV7, AAV8, or AAV9, AAV10, AAV11, preferably AAV9, AAVrh74, AAVrh10. In certain embodiments, AAV capsids may be genetically modified, or capsids may be synthetic, designer capsids that enhance tissue specific or physiologic compartments delivery of a GOI to a specific tissue such as muscle, skeletal muscle, cardiac muscle, smooth muscle, and the like, as described (see, e.g., Zinn and Grimm, High-Throughput Dissection of AAV-Host Interactions: The Fast and the Curious, JMB 430 (17): 2626-2640, 2018; Kotterman and Schaffer, Engineering adeno-associated viruses for clinical gene therapy. Nature Reviews Genetics (2014) 4445-4451, both incorporated herein by reference) and/or detargets certain tissue. Tropism of AAV through pseudotyping, or the mixing of a capsid and genome from different viral serotypes may also be employed. These serotypes are denoted using a slash, so that AAV2/5 indicates a virus containing the genome of serotype 2 packaged in the capsid from serotype 5. Use of these pseudotyped viruses can improve transduction efficiency, as well as alter tropism. For example, pseudotyped AAV2/5 targets myoblasts (Duan et al., Enhancement of muscle gene delivery with pseudotyped adeno-associated virus type 5 correlated with myoblast differentiation. J Virol 75(16):7662-7671, 2001). Other pseudotyped AAV includes AAV2/6. In certain embodiments, In-silico-derived sequences were synthesized de novo and characterized for biological properties relevant to clinical applications. This effort led to the generation of nine functional putative ancestral AAVs and the identification of Anc80, the predicted ancestor of the widely studied AAV serotypes 1, 2, 8, and 9, as a highly potent in vivo gene therapy vector for targeting liver, muscle, and retina (Zinn et al., In Silico Reconstruction of the Viral Evolutionary Lineage Yields a Potent Gene Therapy Vector, Cell Reports 12(6):1056-1068, 2015); Buning et al., Engineering the AAV capsid to optimize vector-host-interactions, Current Opinion in Pharmacology, 24:94-104, 2015).

In certain embodiments, the tropism of the AAV includes skeletal muscle (such as AAV1, AAV6, AAV7, AAV8, or AAV9, preferably AAV9). In certain embodiments, the tropism of the AAV includes cardiac muscle (such as AA1, AAV5, AAV6 AAV8, AAV9, AAVrh74, AAV2-8, AAV2-9,). In certain embodiments, the AAV detargets certain tissue, such as the liver. In certain embodiments, the tropism of the AAV is targeted to certain tissue(s) and detargets certain tissue(s).

In certain embodiments, the gene of interest (GOI) includes a gene responsible for/defective in LGMD2E (limb-girdle muscular dystrophy type 2E), LGMD2D (limb-girdle muscular dystrophy type 2D), LGMD2C (limb-girdle muscular dystrophy type 2C), LGMD2B (limb-girdle muscular dystrophy type 2B), LGMD2L (limb-girdle muscular dystrophy type 2L), LGMD2I (limb-girdle muscular dystrophy type 21), or a gene or coding sequence for NAGLU (α-N-acetylglucosaminidase, for Sanfilippo syndrome or mucopolysaccharidosis type IIIB (MPS IIIB)), sulfamidase or SGSH (for mucopolysaccharidosis type IIIA or MPS IIIA), Factor IX, Factor VIII, Myotubularin 1 (MTM1), Survival of Motor Neuron (SMN, for spinal muscular atrophy or SMA), GalNAc transferase GALGT2, calpain-3 (CAPN-3), acid alpha-glucosidase (GAA, for Pompe disease), alpha-galactosidase A or GLA (for Fabry disease), glucocerebrosidase, dystrophin or microdystrophin.

In certain embodiments, the GOI is a functional equivalent of dystrophin (e.g., a dystrophin minigene encoding a functional micro-dystrophin protein).

In certain embodiments, the GOI is a microdystrophin gene.

In certain embodiments, the microdystrophin gene is one described in U.S. Pat. Nos. 7,906,111; 7,001,761; 7,510,867; 6,869,777; 8,501,920; 7,892,824; WO2016115543; WO 2023/018854, or U.S. Pat. No. 10,166,272 (each and every one incorporated herein by reference). In certain embodiments, the GOI is a microdystrophin gene having the nucleotide sequence of SEQ ID NO: 1 of WO 2023/018854 (incorporated herein by reference).

In certain embodiments, the microdystrophin gene comprises a coding sequence for R16 and R17 spectrin-like repeats for the full length dystrophin protein (such as one described in U.S. Pat. No. 7,892,824).

In certain embodiments, the microdystrophin gene comprises a coding sequence for the R1, R16, R17, R23, and R24 spectrin-like repeats of the full-length dystrophin protein (such as the microdystrophin gene described in PCT/US2016/013733).

In certain embodiments, the microdystrophin gene does not comprise coding sequence for and spectrin repeats of the full-length dystrophin protein other than the SR1, SR16, SR17, SR23, and SR24 repeats (e.g., in that order).

In certain embodiments, the rAAV vectors of the invention are produced in in vitro culture conditions, such as in bioreactors (e.g., 0.5L, 1L, 2L, 3L, 5L, 10L, 20L, 50L, 100L, 250L, 500L, or 1,000L working volume bioreactors), such as a CelliGen Plus packed-bed bioreactor (New Brunswick Scientific) for fed-batch vector production for 3 days post infection.

In certain embodiments, the recombinant replication-defective AAV viral particles are produced as in vitro culture on adherence-dependent cell lines, such as Vero and Vero-derived cell lines or HEK293 cells and HEK293-derived cells, that rely on a solid support. In certain embodiments, the solid support is a tissue culture surface, such as tissue culture dishes, plates, bottles, flasks, cell factory, etc. In certain embodiments, the solid support is a microcarrier, such as Cytodex 1 (GE Healthcare Life Sciences, Piscataway, NJ); a macrocarrier, such as FibraCel (New Brunswick Scientific, Edison, NJ), or a multilayered culture vessel, such as a CellCube (Corning Life Sciences, Lowell, MA) that permit medium perfusion.

In certain embodiments, the recombinant replication-defective AAV viral particles are produced as in vitro culture in eukaryote cells adapted to grow in suspension, such as a suspension culture of the BHK cell line or HEK293 cell line adapted for growth in suspension.

In certain embodiments, the culture supernatant yields in excess of 1×10¹⁰ plaque-forming units (PFU) of rAAV, 1×10¹¹ plaque-forming units (PFU) of rAAV, 1×10¹² plaque-forming units (PFU) of rAAV, 1×10¹³ plaque-forming units (PFU) of rAAV, 1×10¹⁴ plaque-forming units (PFU) of rAAV, or a yield within any range delimited by any pair of the foregoing values, such as between 1×10¹⁰ PFU and 1×10¹⁴ PFU, between 1×10¹¹ PFU and 1×10¹³ PFU, or 1×10¹² PFU and 1×10¹⁴ PFU, for example.

In certain embodiments, vector stock is produced by one or more post processing steps, such as filtration and/or concentration (e.g., depth filtration, dead-end filtration, tangential flow filtration (TFF), and diafiltration), multi-column chromatography purification, final concentration/buffer exchange, etc., to obtain vector stocks with sufficient purity for administration to animals, including human. In certain embodiments, the purification process and the purified vector stock satisfy GMP standard.

In certain embodiments, the titer of the rAAV vector stocks is about 1-2×10⁷ PFU/ml, about 1-2×10⁸ PFU/ml, about 1-2×10⁹ PFU/ml, about 1-2×10¹⁰ PFU/ml, about 1-2×10¹¹ PFU/ml, about 1-2×10¹² PFU/ml, or a titer within any range delimited by any pair of the foregoing values.

In certain embodiments, the total yield of the rAAV vector stock is about 1-25×10¹⁴ total VG of purified rAAV, about 1-10×10¹⁴ total VG of purified rAAV, about 1-5×10¹⁴ total VG of purified rAAV, about 2-4×10¹⁴ total VG of purified rAAV, or a total yield within any range delimited by any pair of the foregoing values.

In certain embodiments, rAAV vectors so produced are further purified from crude cell lysates by ion-exchange chromatography and/or by iodixanol density gradient centrifugation to ensure high final product purity. In certain embodiments, rAAV vectors so produced qualify as a clinical-grade vector batch.

In certain embodiments, the AAV production method of the invention further comprises determining the titer, purity, and/or potency of the rAAV vectors so produced. This may include characterizing the purified rAAV stocks using, e.g., silver staining of SDS-PAGE separation of proteins to determine purity.

### 7. Treatment of Muscular Dystrophy Using AAV

The DT vector of the invention-based system can be used for large scale production of rAAV, which in turn can be used in gene therapy for treating various forms of muscular dystrophy, such as Duchenne's muscular dystrophy (DMD), Myotonic dystrophy, Becker muscular dystrophy (BMD), Limb-girdle muscular dystrophy (LGMD), Facioscapulohumeral muscular dystrophy (FSH), Congenital muscular dystrophy (CDM), Oculopharyngeal muscular dystrophy (OPMD), Distal muscular dystrophy, Emery-Dreifuss muscular dystrophy (EDMD), etc. In certain embodiments, the muscular dystrophy is DMD or BMD.

Thus another aspect of the invention provides a method of treating muscular dystrophy (such as DMD and BMD) in a subject in need thereof, the method comprising administering to the subject a therapeutically effective amount of a recombinant AAV (rAAV) vector encoding a functional version of the gene defective in the muscular dystrophy, such as a microdystrophin gene, wherein the rAAV is produced by the method of the invention using the DT vector of the invention and complementary system.

In certain embodiments, the microdystrophin gene is one described in U.S. Pat. Nos. 7,906,111; 7,001,761; 7,510,867; 6,869,777; 8,501,920; 7,892,824; WO2020/086844; WO2016115543; WO 2023/018854, or U.S. Pat. No. 10,166,272 (all incorporated herein by reference). In certain embodiments, the GOI is a microdystrophin gene having the nucleotide sequence of SEQ ID NO: 1 of WO 2023/018854 (incorporated herein by reference).

In certain embodiments, the microdystrophin gene comprises a coding sequence for the R1, R16, R17, R23, and R24 spectrin-like repeats of the full-length dystrophin protein (such as one described in PCT/US2016/013733).

In certain embodiments, the method further comprises producing the rAAV by the method of the invention using the DT vector system of the invention, prior to administering to the subject the rAAV so produced.

### 8. Treatment of Cardiomyopathy Using AAV

The DT vector of the invention-based system can be used for large scale production of rAAV, which in turn can be used in gene therapy for treating various forms of cardiomyopathy. Cardiomyopathy represents a collection of diverse conditions of the heart muscle and is the second most common cause of heart disease in subjects and medical management of the secondary signs is the only therapeutic option. These diseases have many causes, symptoms, and treatments, and can affect people of all ages and races. When cardiomyopathy occurs, the normal muscle in the heart can thicken, stiffen, thin out, or fill with substances the body produces that do not belong in the heart muscle. As a result, the heart muscle's ability to pump blood is reduced, which can lead to irregular heartbeats, the backup of blood into the lungs or rest of the body, and heart failure. Cardiomyopathy can be acquired or inherited. The cause isn't always known but there is an increasing understanding of the genetic underpinnings of inherited forms of disease. Gene transfer strategies have been shown to ameliorate heart diseases.

Cardiomyopathy is a class of disease of heart muscle that adversely impacts the heart's ability to circulate blood through the cardiovascular system. Various types of cardiomyopathies exist, including dilated cardiomyopathy (DCM), hypertrophic cardiomyopathy (HCM), arrhythmogenic cardiomyopathy (AVC), left ventricular noncompaction cardiomyopathy (LVNC), and restrictive cardiomyopathy (RCM). There are a number of genetic forms of cardiomyopathies, including but not limited to DCM associated with Duchenne and Becker muscular dystrophies. In the case of certain forms of Becker muscular dystrophy, as well as in most cases of Duchenne muscular dystrophy, the cardiomyopathy can ultimately limit the patient's survival. Accordingly, some aspects of the present invention provide AAV or rAAV vectors for delivering transgenes into the heart of a subject, whether directly, systemically or by both methods.

Thus another aspect of the invention provides a method of treating cardiomyopathy in a subject in need thereof, the method comprising administering to the subject a therapeutically effective amount of a recombinant AAV (rAAV) vector encoding a functional version of the gene defective in the cardiomyopathy, genes such as a BAG3 gene, a MYBPC gene, a TMEM43 gene, a TNNT2 gene, an RBM20 gene, an RYR2 gene, a CASQ2 gene, wherein the rAAV is produced by the method of the invention using the DT vector of the invention and complementary system.

Other genes related to various cardiomyopathies include the following:

TABLE 1

| | GENE | GENE NAME |
|---|---|---|
| DCM | ABCC9 | ATP-Binding cassette, sub-family C, member 9 |
| | ACTC | α-Cardiac Actin |
| | ACTN2 | α-Actinin2 |
| | ANKRD1 | Cardiac Ankyrin Repeat, Domain 1 |
| | BAG3 | BCL2-associated athanogene 3 |
| | CSRP3 | Cysteine and Glycine-rich Protein 3 |
| | CTF1 | Cardiotrophin 1 |
| | DES | Desmin |
| | DYS | Dystrophin |
| | DNAJC19 | DnaJ (Hsp40) homolog, subfamily C, member 19 |
| | DSC2 | Desmocollin 2 |
| | DSG2 | Desmoglin 2 |
| | DSP | Desmoplakin |
| | EMD | Emerin |
| | EYA4 | Eyes absent homolog 4 |
| | FHL2 | Four and a half LIM domains 2 |
| | FKTN | Fukutin |
| | FOXD4 | Forkhead box D4 |
| | LAMA4 | α4-Laminin |
| | LAMP2 | Lysosomal-associated membrane protein 3 |
| | LDB3 | LIM-domain binding 3 |
| | LMNA | Lamin A/C |
| | MYBPC3 | Myosin Binding Protein C |
| | MYH6 | β-Myosin Heavy Chain 6 |
| | MYH7 | β-Myosin Heavy Chain 7 |
| | MYPN | Myopalladin |
| | NEBL | Nebulette |
| | NEXN | Nexilin (F actin binding protein) |
| | PLN | Phospholamban |
| | PSEN1 | Presenilin 1 |
| | PSEN2 | Presenilin 2 |
| | RBM20 | RNA binding motif protein 20 |
| | SCN5A | Voltage-gated sodium channel, α subunit |
| | SDHA | Succinate dehydrogenase complex, subunit A, |
| | | flavoprotein |
| | SGCD | δ-Sarcoglycan |
| | SYNE1 | Spectrin repeat containing nuclear protein 1 |
| | SYNE2 | Spectrin repeat containing nuclear protein 2 |
| | TAZ | Tafazzin |
| | TCAP | Titin-cap (Telethonin) |
| | TMPO | Thymopoeitin |
| | TNNC1 | Cardiac Troponin C, type 1 |
| | TNNI3 | Cardiac Troponin I, type 3 |
| | TNNT2 | Cardiac Troponin T, type 2 |
| | TPM1 | α-Tropomyosin 1 |
| | TTN | Titin |
| | TTR | Transthyretin |
| | VCL | Vinculin |
| LVNC | ACTC1 | α-Cardiac Actin |
| | CASQ2 | Cardiac Calsequestrin 2 |
| | DTNA | α-Dystrobrevin |
| | DYS | Dystrophin |
| | GLA | α-Galactosidase |
| | LDB3 | LIM-domain binding 3 |
| | LMNA | Lamin A/C |
| HCM | ACTC | α-Cardiac Actin |
| | ACTN2 | α-Actinin2 |
| | ANKRD1 | Cardiac Ankyrin Repeat, Domain 1 |
| | CAV3 | Caveolin 3 |
| | COX15 | COX 15 homolog, cytochrome C oxidase assembly |
| | | protein |
| | CRYAB | Crystallin αB |
| | CSRP3 | Cysteine and Glycine-rich Protein 3 |
| | GLA | α-Galactosidase |
| | LAMP-2 | Lysomal-Associated Membrane Protein 2 |
| | MYBPC3 | Myosin Binding Protein C |
| | MYH6 | β-Myosin Heavy Chain 6 |
| | MYH7 | β-Myosin Heavy Chain 7 |
| | MYL2 | Myosin Regulatory Light Chain 2, slow |
| | MYL3 | Myosin Light Chain 3, slow |
| | MYLK2 | Myosin Light Chain kinase 2 |
| | MYO6 | Unconventional Myosin VI |
| | MYOZ2 | Myozenin 2 |
| | NEXN | Nexilin (F actin binding protein) |
| | PLN | Phospholamban |
| | PRKAG2 | AMP = activated Protein kinase, |
| | | γ2, non-catalytic subunit |
| | TNNC1 | Cardiac Troponin C, type 1 |
| | TAZ | Tafazzin |
| | TCAP | Titin-cap (Telethonin) |
| | TNNI3 | Cardiac Troponin I, type 3 |
| | TNNT2 | Cardiac Troponin T, type 2 |
| | TPM1 | α-Tropomyosin 1 |
| | TTN | Titin |
| | TTR | Transthyretin |
| | VCL | Vinculin |
| RCM | ACTC1 | α-Cardiac Actin |
| | BAG3 | BCL2-associated athanogene 3 |
| | DES | Desmin |
| | GLA | α-Galactosidase |
| | MYH7 | β-Myosin Heavy Chain 7 |
| | MYL2 | Myosin Regulatory Light Chain 2, slow |
| | MYL3 | Myosin Light Chain 3, slow |
| | MYPN | Myopalladin |
| | TNNI3 | Cardiac Troponin I, type 3 |
| | TNNT2 | Cardiac Troponin T, type 2 |
| | TPM1 | α-Tropomyosin 1 |
| | TTN | Titin |
| | TTR | Transthyretin |
| AVC | CTNNA3 | αT-Catenin |
| | DES | Desmin |
| | DNAJC19 | DnaJ (Hsp40) homolog, subfamily C, member 19 |
| | DSC2 | Desmocollin 2 |
| | DSG2 | Desmoglin 2 |
| | DSP | Desmoplakin |
| | JUP | Junctional Plakoglobin |
| | LMNA | Lamin A/C |
| | PKP2 | Plakophilin 2 |
| | PKP4 | Plakophilin 4 |
| | RYR2 | Ryanodine receptor 2 |
| | STRN | Striatin |
| | TGFβ3 | Transforming growth factor-β3 |
| | TMEM43 | Transmembrane 43 |
| | TTN | Titin |

### EXAMPLES

### Example 1 AAV Production Using Dual Transfection System

### Transfection and Sampling

About 24 hours prior to transfection, HEK293 cells (Expi293™ working cell bank) were inoculated in 50 mL fresh Freestyle™ F17 media (supplemented with 4 mM Glutamax, 0.1% F68) in a shake flask at 1.5×10⁶ (1.5E6) cells/mL concentration. On the day of transfection, the target cell density is 2.5E6-3.5E6 cells/mL. A plasmid cocktail was prepared by adding plasmids to 15 mL of un-supplemented Freestyle™ F17 media in a tube, mixing, adding PEI (Polyethylenimine) transfection reagent, incubating the mixture for 5 minutes, and mix the cocktail again by inverting the tube. Each plasmid cocktail was added to the cells in each shake flask. The cells and plasmid cocktail were gently mixed together and then incubated at 110 RPM, 5% CO₂ and 37° C. About 2-20 hours post transfection, the plasmids were neutralized with 50 μL of Gibco™ anti-clumping agent.

Samples were collected from the flasks at 2-4 days post transfection, and the cells were lysed by Triton-X treatment. After centrifugation, the supernatant of the centrifuged lysed cells were collected and stored at −80° C. for ddPCR analysis.

### AAV9 Production Using Dual Transfection System

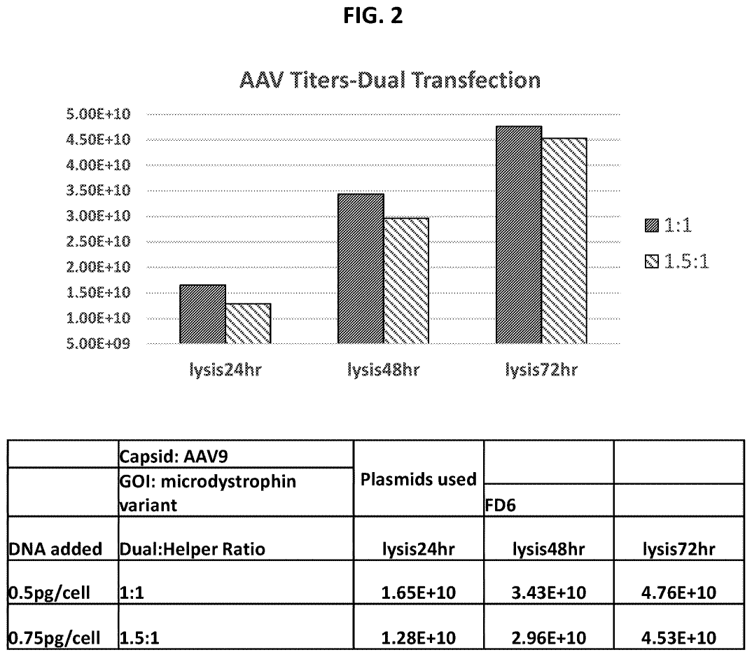

Dual plasmid comprising RepCap genes for AAV9 and a microdystrophin variant as the gene-of interest (GOI) were transfected into HEK293 cells with helper plasmid at a Dual: Helper (w/w) ratio of 1:1 or 1.5:1 as described above. Experimental details and results are depicted in FIG. 2.

As shown in FIG. 2, dual transfection using the dual vector of the invention comprising both RepCap and GOI sequences in one plasmid were sufficient at producing AAV particles.

### Example 2 Comparison Between Dual Transfection and Triple Transfection Methods-Titer

### Example 2A

AAV9 and AAV-SLB101 production using the dual transfection (DT) and triple transfection (TT) methods were carried out using the transfection method as described above, in Example 1, with Dual:Helper (w/w) ratio of 1:1 and RepCap:GOI:Helper (w/w) ratio of 1:1:1, respectively. The amount of each plasmid added to HEK293 cells for transfection is 0.25 μg/cell. AAV9 and AAV-SLB101 produced were determined at 24, 48 and 72 hours post transfection as measured by ddPCR.

It should be understood that, although AAV9 and SLB101 were used in this example to demonstrate the effectiveness of the dual plasmid system of the present invention useful for AAV production, and the surprisingly higher titer compared to the triple transfection method, the capsids that can be used in such dual transfection vectors/plasmids are without limit.

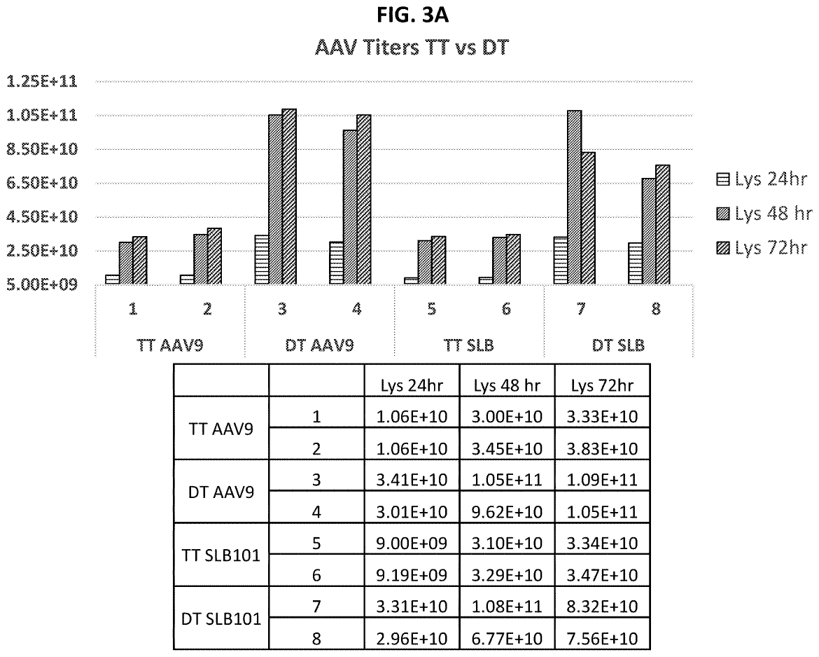

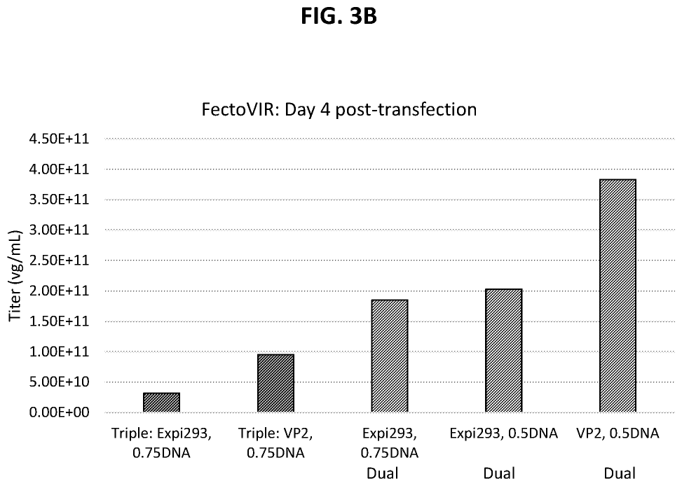

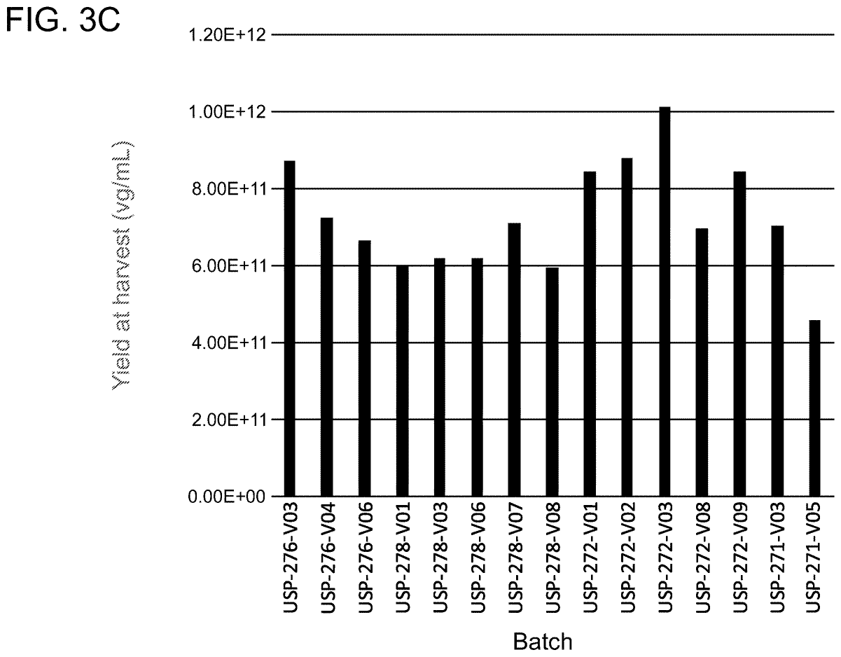

Viral production was compared between the two transfection methods, and the results are shown in FIG. 3A.

Surprisingly, both AAV9 and AAV-SLB101 viral titers were at least 2- to 3-fold (i.e., 200-300%) higher in the dual transfection method compared to the triple transfection method, at all measured time points. See FIG. 3A.

This experiment suggests that the dual transfection method using a DT-vector of the invention (comprising both a GOI and Rep/Cap sequences) generates surprisingly higher viral yield compared to the conventional triple transfection method (where GOI, RepCap and Helper) are encoded by 3 separate plasmids.

### Example 2B

Vectors comprising AAV-SLB101 with microdystrophin GOI (AV-SLB101-uDys) were produced by both dual transfection system of the invention and triple transfection system using an AMBR® 15 cell culture, a high throughput, automated advanced microbioreactor system for 48 parallel cultivations at the 10-15 mL microbioreactor scale. and according to the parameters in Table 2 below.

TABLE 2

| | | | | Complex | PEI:DNA | DNA amount |
|---|---|---|---|---|---|---|
| DNA Type | Cell Line | Plasmid Ratio | Media | Time | ratio | (pg/vc) |
| Triple | EXPI | 1.5:1:1 | F17 | 60 mins | 1 | 0.75 |
| | | GOI:RC:Helper | | | | |
| Triple | VP | 1.5:1:1 | Expi293 | 15 mins | 2.5 | 0.75 |
| | | GOI:RC:Helper | | | | |
| Dual | EXPI | 1:1 | F17 | 30 mins | 1 | 0.75 |
| Dual | EXPI | 1:1 | F17 | 30 mins | 1 | 0.5 |
| Dual | VP | 1:1 | F17 | 30 mins | 1 | 0.5 |

FIG. 3B shows the titer for each sample Day 4 post-transfection using Fecto VIR. Viral titers using triple transfection (TT) achieved only from under 3.2E10 vg/mL to at most, less than 1E11 vg/mL. Yet, viral titers using the dual transfection (DT) method of the invention ranged from the lowest, 1.85E11 vg/mL up to 3.83E11 vg/mL, an increase of almost 100% to almost 400% over the titers from the triple transfection methods.

### Example 2C

AAV vectors (AAV-SLB101-uDys) in Example 2B were produced on a larger scale using 2L bioreactors according to the parameters in Table 3 below.

TABLE 3

| Batch Generator | Scale | Media | Quench |
|---|---|---|---|
| USP-276-V02 | 2 L | Expi293 | Yes |
| USP-276-V03 | 2 L | Expi293 | Yes |
| USP-276-V04 | 2 L | Expi293 | Yes |
| USP-276-V06 | 2 L | Expi293 | Yes |
| USP-278-V01 | 2 L | F17 | No |
| USP-278-V03 | 2 L | F17 | No |
| USP-278-V06 | 2 L | F17 | No |
| USP-278-V07 | 2 L | Expi293 | No |
| USP-278-V08 | 2 L | Expi293 | No |
| USP-272-V01 | 2 L | Expi293 | Yes |
| USP-272-V02 | 2 L | Expi293 | Yes |
| USP-272-V03 | 2 L | Expi293 | Yes |
| USP-272-V08 | 2 L | Expi293 | Yes |
| USP-272-V09 | 2 L | Expi293 | Yes |
| USP-271-V03 | 2 L | Expi293 | Yes |
| USP-271-V05 | 2 L | Expi293 | Yes |

Results are shown in FIG. 3C in which all samples achieved from at least 4.55E11 vg/mL, and reached as high as 1E12 vg/mL (FIG. 3C). As compared to FIG. 3B, yield translated well from 10-15 mL microbioreactor (Example 2B) to the 2L bioreactor, supporting scalability of the dual transfection system of the invention.

### Example 3 Comparison Between Dual Transfection and Triple Transfection Methods-Mis-Packaging Rates

Using the AMBR® 15 cell culture (see above), Kanamycin titer was measured and Kanamycin titer relative to GOI titer was calculated and compared between the DT system of the invention and the triple transfection system, using two commercially available transfection reagents—the Gibco AAV-MAX Transfection Kit (ThermoFisher Scientific), and the FECTOVIR®-AAV transfection reagent for large scale rAAV manufacturing (Polyplus, NY).

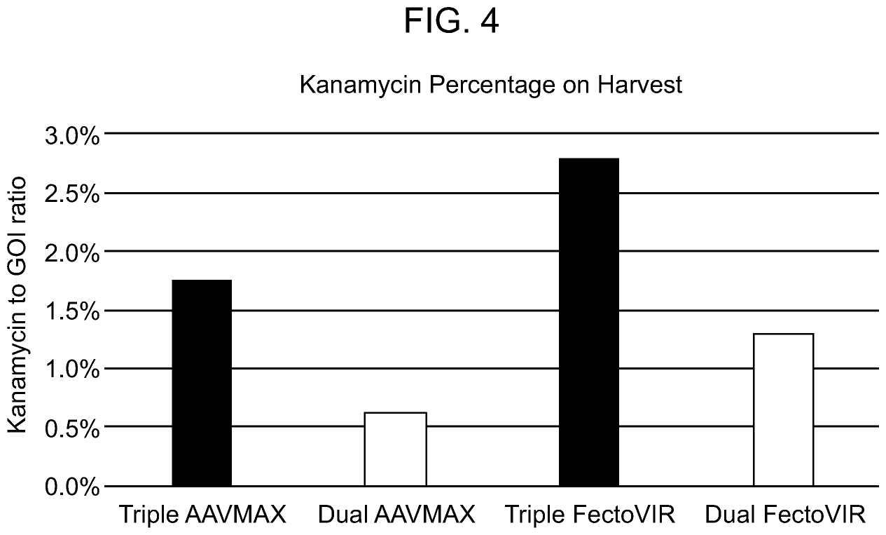

FIG. 4 shows that Kanamycin titer on harvest was much lower using the DT plasmid system of the invention, compared to the TT system, based on the ratio of Kanamycin to GOI by ddPCR using both AAV-MAX and FectoVIR as transfection reagents.

### Example 4 Comparison of GOI Titers and p5 Promoter Packaging to GOI

GOI titers and P5:GOI as a percentage were both measured by ddPCR in the DT plasmid system of the invention with a P5 promoter, and a modified P5 promoter.

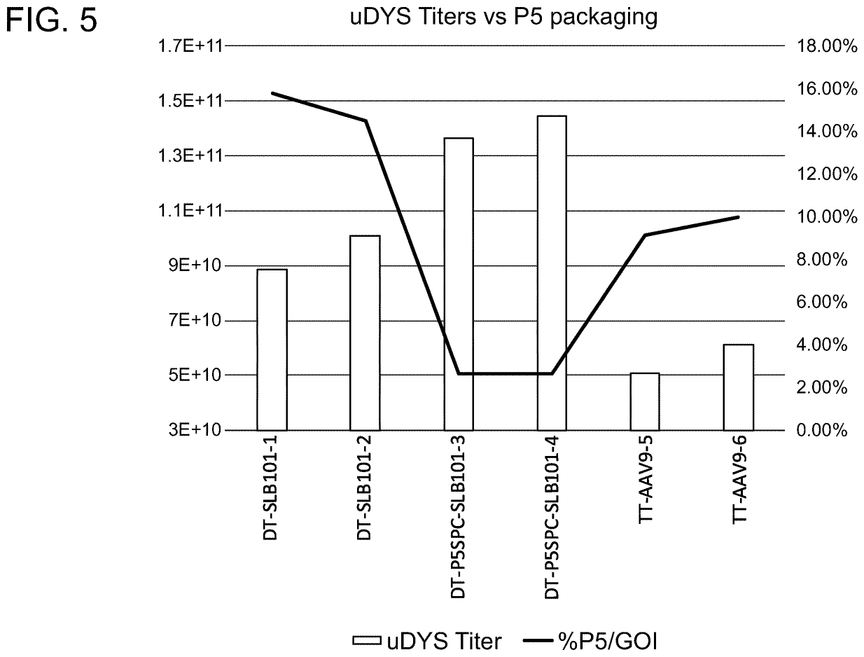

For the DT plasmid system with modified P5 promoter, wild-type AAV2 P5 promoter in the RepCap cassette was modified by inserting an exogenous 5 nucleotide spacer sequence between the REP binding site and YY1 binding site of the wild-type P5 promoter, P5SPC promoter. The DT plasmid of the invention comprising the P5SPC promoter driving the transcription of the RepCap cassette was designated as “P5SPC-SLB101” (DT-P5SPC-SLB101-1 and DT-P5SPC-SLB101-2) in FIG. 5. Mis-packaging of DNA upstream of the P5 promoter in the plasmid vector (as exemplified by the KanR coding sequence) for DT-P5SPC-SLB101-1 and DT-P5SPC-SLB101-2 was significantly reduced as compared to other samples, while maintaining recombinant AAV titers.

FIG. 5, shows that Kanamycin relative to GOI was lower (about 2.6%) for DT plasmids having the P5SPC promoter (DT-P5SPC-SLB101), vs. DT plasmids having the wild-type P5 promoter (DT-SLB101, about 15%), using both AAV-MAX and Fecto VIR transfection reagents (see DT-SLB101-1 and DT-SLB101-2, vs. DT-P5SPC-SLB101-3 and DT-P5SPC-SLB101-4). Also see the data summarized in table 4 below.

TABLE 4

| | P5 | P5 SPC | μDYS | % |
|---|---|---|---|---|
| AAV Prep | Titer | Titer | Titer | P5/GOI |
| DT-SLB101-1 | 1.40E+10 | | 8.84E+10 | 15.80% |
| DT-SLB101-2 | 1.46E+10 | | 1.01E+11 | 14.48% |
| DT-P5SPC-SLB101-3 | 2.36E+09 | 3.58E+09 | 1.36E+11 | 2.63% |
| DT-P5SPC-SLB101-4 | 2.17E+09 | 3.79E+09 | 1.45E+11 | 2.62% |
| TT-AAV9-5 | 4.64E+09 | | 5.07E+10 | 9.15% |
| TT-AAV9-6 | 6.11E+09 | | 6.13E+10 | 9.97% |

Meanwhile, titer for the AAV virus produced using the DT plasmids with the P5SPC promoter and the DT plasmids with wild-type P5 promoter, using both AAV-MAX and Fecto VIR transfection reagents were significantly higher than titers for AAV produced using triple transfection (FIG. 5).

1. A recombinant DNA vector comprising:
(a) a gene-of-interest (GOI) flanked by a 5′ adeno-associated virus (AAV) ITR sequence and a 3′ ITR sequence;
(b) a coding sequence for an AAV Rep compatible with said 5′ AAV ITR and said 3′ AAV ITR, optionally, said coding sequence for the AAV Rep is under the transcriptional control of an AAV P5 promoter positioned downstream or 3′ to the GOI; and,
(c) a coding sequence for an AAV Cap;
wherein expression of said AAV Rep, in a host cell comprising AAV helper genes, is sufficient to package an AAV vector genome (vg) comprising the GOI flanked by the 5′- and 3-ITR sequences into an AAV capsid comprising said AAV Cap, with the proviso that the recombinant DNA vector is not a Herpes Simplex Virus (HSV) vector.

2. The vector of claim 1, which is a plasmid.

3. A vector comprising:
(a) a gene-of-interest (GOI) flanked by adeno-associated virus (AAV) ITRs;
(b) a coding sequence for an AAV Rep compatible with said ITRs, optionally, said coding sequence for the AAV Rep is under the transcriptional control of an AAV P5 promoter positioned downstream or 3′ to the GOI; and,
(c) a coding sequence for an AAV Cap;
wherein the vector is not a Herpes Simplex Virus (HSV) vector.

4. A plasmid comprising:
(a) a gene-of-interest (GOI) flanked by (AAV) ITRs;
(b) a coding sequence for an AAV Rep compatible with said (AAV) ITRs, optionally, said coding sequence for the AAV Rep is under the transcriptional control of an AAV P5 promoter positioned downstream or 3′ to the GOI; and,
(c) a coding sequence for an AAV Cap.

5. A plasmid comprising:
(a) a gene-of-interest (GOI) flanked by a 5′ adeno-associated virus (AAV) ITR sequence and a 3′ ITR sequence;
(b) a coding sequence for an AAV Rep compatible with said 5′ AAV ITR and said 3′ AAV ITR, optionally, said coding sequence for the AAV Rep is under the transcriptional control of an AAV P5 promoter positioned downstream or 3′ to the GOI; and,
(c) a coding sequence for an AAV Cap;
wherein expression of said AAV Rep, in a host cell comprising AAV helper genes, is sufficient to package an AAV vector genome (vg) comprising the GOI flanked by the 5′- and 3-ITR sequences into an AAV capsid comprising said AAV Cap.

6. A two-plasmid system comprising a vector plasmid and a helper plasmid, wherein the vector plasmid comprises a GOI, a rep gene encoding functional Rep proteins, and a cap gene encoding functional Cap proteins.

7. The vector of any of claims 1-6, wherein the GOI is within a pro-AAV cassette comprising the GOI operably linked to a promoter.

8. The vector of claim 7, wherein the pro-AAV cassette further comprises:
(1) an enhancer that promotes the transcription of the GOI from the promoter;
(2) a 5′ UTR;
(3) a Kozak sequence;
(4) a heterologous intron that promotes transcription and/or translation of the GOI;
(5) a 3′ UTR;
(6) a WPRE sequence; and/or
(7) a polyA signal sequence.

9. The vector of any one of claims 1-8, wherein the coding sequence for the AAV Rep and the coding sequence for the AAV Cap are within a RepCap cassette comprising an operably-linked RepCap promoter (such as the AAV P5 promoter).

10. The vector of claim 9, wherein the RepCap cassette and the pro-AAV cassette:
i) are immediately adjacent to each other (e.g., with substantially no intervening polynucleotide sequence);
ii) are not immediately adjacent to each other;
iii) have the same transcription direction; or,
iv) have opposite transcription direction.

11. The vector of claim 10, further comprising a bacterial replication Ori gene, a selection marker (such as an antibiotic resistance gene, e.g., KanR or AmpR) under the transcriptional control of a selection marker promoter (such as a bacterial promoter).

12. The vector of any one of claims 1-11, wherein said GOI is a functional equivalent of dystrophin (e.g., a dystrophin minigene encoding a functional micro-dystrophin protein).

13. The vector of any one of claims 1-12, wherein said GOI includes a gene responsible for/defective in LGMD2E (limb-girdle muscular dystrophy type 2E), LGMD2D (limb-girdle muscular dystrophy type 2D), LGMD2C (limb-girdle muscular dystrophy type 2C), LGMD2B (limb-girdle muscular dystrophy type 2B), LGMD2L (limb-girdle muscular dystrophy type 2L), LGMD2I (limb-girdle muscular dystrophy type 2I), or a gene or coding sequence for NAGLU (α-N-acetylglucosaminidase, for Sanfilippo syndrome or mucopolysaccharidosis type IIIB (MPS IIIB)), sulfamidase or SGSH (for mucopolysaccharidosis type IIIA or MPS IIIA), Factor IX, Factor VIII, Myotubularin 1 (MTM1), Survival of Motor Neuron (SMN, for spinal muscular atrophy or SMA), GalNAc transferase GALGT2, calpain-3 (CAPN-3), acid alpha-glucosidase (GAA, for Pompe disease), alpha-galactosidase A or GLA (for Fabry disease), glucocerebrosidase, dystrophin or microdystrophin.

14. The vector of any one of claims 1-13, wherein the GOI is a microdystrophin gene (e.g., one described in U.S. Pat. Nos. 7,906,111; 7,001,761; 7,510,867; 6,869,777; U.S. Pat. Nos. 8,501,920; 7,892,824; WO2016115543; WO 2023/018854, or U.S. Pat. No. 10,166,272).

15. The vector of claim 14, wherein the microdystrophin gene comprises a coding sequence for R16 and R17 spectrin-like repeats for the full length dystrophin protein (such as one described in U.S. Pat. No. 7,892,824).

16. The vector of claim 15, wherein the microdystrophin gene comprises a coding sequence for the R1, R16, R17, R23, and R24 spectrin-like repeats of the full-length dystrophin protein (such as the microdystrophin gene described in WO2016115543 or SEQ ID NO: 1 of WO 2023/018854).

17. The vector of any one of claims 1-16, wherein said 5′ and 3′ AAV ITR sequences flanking said GOI are both from AAV1, AAV2, AAV3A, AAV3B, AAV4, AAV5, AAV6, AAV7, AAV8, AAV9, AAV10, AAV11, AAV12, AAV13, AAVrh74, or AAV-DJ.

18. The vector of claim 17, wherein the tropism of the AAV include skeletal muscle (such as AAV1, AAV6, AAV7, AAV8, or AAV9, preferably AAV9).

19. The vector of any one of claims 1-18, wherein said AAV ITR, said AAV Rep, and said AAV Cap are from the same or different AAVs.

20. The vector of claim 19, wherein said AAV ITR is AAV2 ITR, said AAV Rep is Rep2 from AAV2, and said AAV Cap is Cap9 from AAV9 or a derivative thereof.

21. The vector of any one of claims 1-20, wherein said coding sequence for AAV Rep and Cap proteins is under the transcriptional control of a promoter, such as an AAV P5 promoter, an upstream HSV promoter, a modified P5 promoter lacking RBE (Rep-Binding Element), an HPV P97 promoter containing a REP binding site and a transcription start site-localized YY1 binding site, or a ubiquitous promoter (such as CMV promoter, EF1a promoter, CAG promoter, CB promoter etc).

22. The vector of claim 21, wherein the modified P5 promoter is a recombinant P5 promoter comprising a REP binding site and a transcription start site-localized Ying-Yang 1 (YY1) binding site, and wherein said modified P5 promoter comprises an exogenous spacer sequence inserted between the REP binding site and YY1 binding site; optionally, the spacer is 5 nucleotides to 100 nucleotides in length (e.g., about 5 nts).

23. The vector of any one of claims 1-22, wherein said AAV helper genes comprise adenoviral, herpesviral, or papillomaviral genes for AAV packaging (such as E1A, E1B, E2A, E4 and VA RNA), optionally operably linked to a promoter as one transcriptional unit.

24. A composition or a kit, comprising (1) the vector of any one of claims 1-23, and (2) a helper plasmid.

25. The composition or kit of claim 24, wherein the helper plasmid comprises a helper virus gene that supports AAV packaging.

26. The composition or kit of claim 25, wherein the helper virus gene comprises:
(i) an adenovirus gene, optionally an Adenovirus 5 or Adenovirus 2 gene; and/or
(ii) a VA nucleic acid encoding functional VA RNA I and II, an E2A gene encoding a functional E2A protein, and an E4 gene encoding a functional E4 protein.

27. A host cell comprising the vector or the two-plasmid system of any one of claims 1-23, or the composition of any one of claims 24-26.

28. The host cell of claim 27, which is a HEK293 cell (such as an Expi293F cell), a VP2 cell, a Vero cell, an HUH7 cell, a HepG2 cell, a HeLa cell, an A549 cell, a BHK cell, or an insect cell (such as Sf9).

29. A method of propagating/amplifying/producing a recombinant replication-defective AAV viral particle encapsidating the GOI of any one of claims 1-23, the method comprising: introducing the vector of any one of claims 1-23 into a host cell, prior to, concurrently with, or subsequent to introducing said AAV helper genes of any one of claims 1-23 to said host cell, thereby propagating/amplifying/producing the recombinant replication-defective AAV viral particle.

30. The method of claim 29, further comprising harvesting the recombinant replication-defective AAV viral particle from the host cell.

31. The method of claim 29 or 30, wherein the host cell is a HEK293 cell (such as an Expi293F cell), a VP2 cell, a Vero cell, an HUH7 cell, a HepG2 cell, a HeLa cell, an A549 cell, a BHK cell, or an insect cell (such as Sf9).

32. The method of any one of claims 29-31, wherein the recombinant DNA vector is introduced to the host cell by transient transfection.
